(19) United States
(12) Patent Application Publication
(10) Pub. No.: US 2019/0077851 A1
(43) Pub. Date: Mar. 14, 2019

(54) METHODS OF USING ANTI-ALPHA TOXIN ANTIBODY

(71) Applicants: Bruno FRANCOIS, Limoges (FR); MEDIMMUNE, LLC, Gaithersburg, MD (US)

(72) Inventors: Hasan JAFRI, Gaithersburg, MD (US); Terramika BELLAMY, Gaithersburg, MD (US); Susan COLBERT, Gaithersburg, MD (US); Brian BISHOP, Gaithersburg, MD (US); Gabriel ROBBIE, Gaithersburg, MD (US); Mark ESSER, Gaithersburg, MD (US); Bret SELLMAN, Gaithersburg, MD (US); Kathryn JENSEN, Gaithersburg, MD (US); Kellie RYAN, Gaithersburg, MD (US); Xiang-Qing YU, Gaithersburg, MD (US); Bruno FRANCOIS, Limoges (FR)

(21) Appl. No.: 15/769,221

(22) PCT Filed: Oct. 27, 2016
(86) PCT No.: PCT/US2016/059070

§ 371 Date: Apr. 18, 2018

Related U.S. Application Data
(60) Provisional application No. 62/248,769, filed on Oct. 30, 2015.

(51) Int. Cl. C07K 16/12; A61P 31/04; A61P 11/00

(52) U.S. Cl. CPC C07K 16/1271; A61P 31/04; C07K 2317/565; C07K 2317/94; C07K 2317/76; A61P 11/00

(57) ABSTRACT

Provided herein are methods of preventing and treating a bacterial infection, e.g., a Staphylococcus aureus infection, in a patient, comprising administering to the patient an effective amount of an anti-alpha toxin antibody or an antigen-binding fragment thereof, such as MEDI4893.

### REFERENCE TO SEQUENCE LISTING SUBMITTED ELECTRONICALLY

The content of the electronically submitted sequence listing in ASCII text file ATOX-200P1_SeqList.txt (Size: 12,427 bytes; and Date of Creation: Oct. 30, 2015) filed with the application is incorporated herein by reference in its entirety.

### BACKGROUND OF THE INVENTION

### Field of the Invention

The technology relates to the use of anti-alpha toxin antibodies or antigen-binding fragments thereof for the treatment and prevention of infections.

### Background of the Invention

Staphylococcus aureus (S. aureus) is a Gram-positive, facultatively aerobic, clump-forming cocci bacterium that commonly colonizes the nose and skin of healthy humans. Approximately 20-30% of the population is colonized with S. aureus at any given time. Mucosal and epidermal barriers (skin) normally protect against S. aureus infections, but opportunistic S. aureus infections can become serious, causing a variety of diseases or conditions, non-limiting examples of which include bacteremia, cellulitis, eyelid infections, food poisoning, joint infections, skin infections, scalded skin syndrome, toxic shock syndrome, pneumonia, osteomyelitis, endocarditis, meningitis, and abscess formation. Infections with S. aureus are associated with increased morbidity and mortality.

Bacterial pneumonia occurring within the hospitalised or intensive care unit (ICU) population is a clinically significant and serious disease that contributes significantly to morbidity and mortality. This constitutes the second leading type of nosocomial infection and the leading cause of death from nosocomial infection in the United States of America (USA; Spellberg and Talbot, “Recommended design features of future clinical trials of antibacterial agents for hospital-acquired bacterial pneumonia and ventilator-associated bacterial pneumonia,” Clin Infect Dis. 51(Suppl 1):S150-70 (2010)). S. aureus is the primary cause of nosocomial pneumonia. A recent study of European ICUs reported that 23% of mechanically ventilated ICU patients developed pneumonia caused by S. aureus, with over half caused by methicillin-resistant Staphylococcus aureus (MRSA) (Esperatti et al., “Nosocomial pneumonia in the intensive care unit acquired by mechanically ventilated versus nonventilated patients,” Am J Respir Crit Care Med 182:1533-9 (2010)). A study of morbidity and mortality associated with S. aureus pneumonia in ICUs across Europe and Latin America found a mean duration for mechanical ventilation of 15.9 days (range 11 to 30.3 days), mean ICU mortality of 21.6% (range 10% to 60%), and mean Acute Physiology and Chronic Health Evaluation (APACHE) score of 18.6 (range 15.2 to 22) for patients with S. aureus pneumonia (Rello et al., “Differences in hospital- and ventilator-associated pneumonia due to Staphylococcus aureus (methicillin-susceptible and methicillin-resistant) between Europe and Latin America: a comparison of the EUVAP and LATINVAP study cohorts,” Med Intensiva 37:241-7 (2013)).

S. aureus pneumonia among mechanically ventilated intubated ICU patients is associated with significant healthcare-associated costs. Restrepo and colleagues (Restrepo et al., “Economic burden of ventilator-associated pneumonia based on total resource utilization,” Infect Control Hosp Epidemiol. 31:509-15 (2010)) reported median incremental hospital costs of S. aureus pneumonia compared to non-pneumonia controls to be $101,660 when examined between 2002 and 2006. A more recent analysis of a USA claims database of privately insured patients admitted to an ICU between 2006 and 2012 suggests that S. aureus pneumonia was associated with a mean excess or incremental cost of approximately $100,000 compared to the control intubated ICU patients.

Further complicating the morbidity and mortality described above, available therapy for treating S. aureus pneumonia cases is often limited due to antibiotic-resistant strains or the patient's intolerance to treatment. Even with the introduction of new antibiotics against S. aureus, continuing emergence of resistance requires new approaches to address the existing, and potentially expanding, unmet medical need for preventing S. aureus disease.

Two major variables associated with an increased risk of S. aureus pneumonia are respiratory colonisation with S. aureus and the prolonged need for mechanical ventilation with endotracheal intubation (>48 hours). The rate of S. aureus pneumonia among intubated patients colonised with S. aureus has been reported to be up to 35% (Sirvent et al., “Tracheal colonisation within 24 h of intubation in patients with head trauma: risk factor for developing early-onset ventilator-associated pneumonia,” Intensive Care Med. 26:1369-72 (2000); Ewig et al., “Bacterial colonization patterns in mechanically ventilated patients with traumatic and medical head injury,” Am J Respir Crit Care Med. 159:188-98 (1999)). A recent study by Mullins and colleagues (Mullins et al., “Methicillin-resistant Staphylococcus aureus (MRSA) surveillance culture as a predictor for MRSA-ventilator associated pneumonia” (poster). Infectious Diseases Society of America (IDSA) Annual Meeting, Poster abstract 349, San Francisco, Calif., (2013)) reported a 62.2% S aureus pneumonia rate among mechanically ventilated intubated patients colonised with MRSA.

During infection, S. aureus releases a number of toxins, and S. aureus alpha toxin (AT) is the most prevalent virulence factor causing tissue invasion and necrosis (Wilke and Wardenburg, “Role of a disintegrin and metalloprotease 10 in Staphylococcus aureus alpha-hemolysin-mediated cellular injury,” Proc Natl Acad Sci USA. 107(30):13473-8 (2010). The pivotal role of AT in S. aureus pathogenesis is supported by animal models (dermonecrosis, pneumonia, sepsis, endocarditis, and mastitis) (Bramley et al., “Roles of alpha-toxin and beta-toxin in virulence of Staphylococcus aureus for the mouse mammary gland,” Infect Immun. 57(8):2489-94 (1989); Bayer et al., “Hyperproduction of alpha-toxin by Staphylococcus aureus results in paradoxically reduced virulence in experimental endocarditis: a host defense role for platelet microbicidal proteins,” Infect Immun. 65(11):4652-60 (1997); Bubeck Wardenburg et al., “Panton-Valentine leukocidin is not a virulence determinant in murine models of community-associated methicillin-resistant Staphylococcus aureus disease,” J Infect Dis. 198(8): 1166-70 (2008); Kobayashi et al., “Comparative analysis of USA300 virulence determinants in a rabbit model of skin and soft tissue infection,” J Infect Dis. 204(6):937-41 (2011); and Powers et al., “ADAM10 mediates vascular injury induced by Staphylococcus aureus α-hemolysin,” J Infect Dis. 206(3):352-6 (2012)) and by observational studies in humans in which the presence of anti-AT antibodies during severe infections was associated with improved outcome (Adhikari et al., “Lower antibody levels to Staphylococcus aureus exotoxins are associated with sepsis in hospitalized adults with invasive S aureus infections,” J Infect Dis. 206(6):915-2 (2012); Jacobsson et al., “Antibody responses in patients with invasive Staphylococcus aureus infections,” Eur J Clin Microbiol Infect Dis. 29:715-25 (2010); and Ruotsalainen et al., “Methicillin-sensitive Staphylococcus aureus bacteraemia and endocarditis among injection drug users and nonaddicts: host factors, microbiological and serological characteristics,” J Infect. 56:249-56 (2008)).

Antibiotics are the only intervention available for treating S aureus diseases. Despite the introduction of new antibiotics against S. aureus, emergence of resistance requires new approaches for combatting S. aureus diseases. While prevention of healthcare-associated infections caused by S. aureus is an important public health goal, no vaccines or passive immunisation therapies are available. Prevention currently focuses on infection control practices and limited prophylactic use of antibiotics (e.g., presurgery). Topical decolonisation regimens have been proposed for S. aureus carriers, given that nasal carriage is a risk factor for hospital-acquired infection (Muñoz et al., “Nasal carriage of S. aureus increases the risk of surgical site infection after major heart surgery,” J Hosp Infect. 68((1):25-31 (2008); and Bode et al., “Preventing surgical-site infections in nasal carriers of Staphylococcus aureus,” N Engl J Med. 362(1):9-17 (2010)). However, decolonisation efforts have not been consistently effective and are not universally implemented (Kluytmans et al., “Reduction of surgical-site infections in cardiothoracic surgery by elimination of nasal carriage of Staphylococcus aureus,” Infect Control Hosp Epidemiol. 17(12):780-5 (1996); Perl et al., “Intranasal mupirocin to prevent postoperative Staphylococcus aureus infections,” N Engl J Med. 346(24): 1871-7 (2002); and Kalmeijer et al., “Surgical site infections in orthopedic surgery: the effect of mupirocin nasal ointment in a double-blind, randomized, placebo-controlled study,” Clin Infect Dis. 35(4):353-8 (2002)). Thus, there is a need for novel strategies for preventing and treating bacterial infections.

### BRIEF SUMMARY OF THE INVENTION

The present disclosure provides methods of using anti-alpha toxin antibodies or antigen-binding fragments thereof, for example, for the treatment and prevention of infections.

In some embodiments, a method of treating or preventing a nosocomial infection in a subject comprises administering 2000 to 5000 milligrams of an anti-alpha toxin antibody or antigen-binding fragment thereof to the subject, wherein the antibody or antigen-binding fragment thereof comprises a set of Complementarity-Determining Regions (CDRs): HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6.

In some embodiments, a method of treating or preventing a nosocomial infection in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6; wherein the serum target level of the antibody or antigen-binding fragment thereof (e.g., MEDI4893) is in a range of about 100 μg/ml to about 1000 μg/ml, about 200 μg/ml to about 500 μg/ml, about 250 μg/ml to about 450 μg/ml, about 200 μg/ml to about 300 μg/ml, about 211 μg/ml to about 1000 μg/ml, about 211 μg/ml to about 500 μg/ml, about 211 μg/ml to about 400 μg/ml, about 211 μg/ml to about 300 μg/ml, or at about 200 μg/ml, at about 210 μg/ml, at about 220 μg/ml, at about 230 μg/ml, at about 240 μg/ml, at about 250 μg/ml, at about 300 μg/ml, or at least 211 μg/ml.

In some embodiments, a method of treating or preventing a nosocomial infection in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6; wherein the administration results in a serum level of the antibody or antigen-binding fragment (e.g., MEDI4893) of at least 211 μg/ml.

In some embodiments, a method of decreasing the severity of a nosocomial infection in a subject comprises administering 2000 to 5000 milligrams of an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6.

In some embodiments, a method of decreasing the severity of a nosocomial infection in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6; wherein the serum target level of the antibody or antigen-binding fragment thereof (e.g., MEDI4893) is at least 211 μg/ml.

In some embodiments, a method of decreasing the severity of a nosocomial infection in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6; wherein the administration results in a serum level of the antibody or antigen-binding fragment (e.g., MEDI4893) of at least 211 μg/ml.

In some embodiments of the methods provided herein, the infection is a Staphylococcus aureus (S. aureus) infection. In some embodiments, the infection is selected from the group consisting of pneumonia, bacteremia, bone infection, joint infection, deep skin infection, tissue infection, meningitis, or endocarditis. In some embodiments, the infection is pneumonia. In some embodiments, the infection is ventilator associated pneumonia. In some embodiments, the infection is pneumonia following extubation.

In some embodiments, a method of treating or preventing pneumonia in a subject comprises administering 2000 to 5000 milligrams of an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein. HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6.

In some embodiments, a method of treating or preventing pneumonia in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6, wherein the serum target level of the antibody or antigen-binding fragment thereof (e.g., MEDI4893) is at least 211 μg/ml.

In some embodiments, a method of treating or preventing pneumonia in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6, wherein the administration results in a serum level of the antibody or antigen-binding fragment (e.g., MEDI4893) of at least 211 μg/ml.

In some embodiments, a method of decreasing the severity of pneumonia in a subject comprises administering 2000 to 5000 milligrams of an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6.

In some embodiments, a method of decreasing the severity of pneumonia in a human subject comprises administering an anti-alpha toxin antibody or antigen-binding fragment thereof to the subject, wherein the antibody or antigen-binding fragment thereof comprises a set CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6, wherein the serum target level of the antibody or antigen-binding fragment thereof (e.g., MEDI4893) is at least 211 μg/ml.

In some embodiments, a method of decreasing the severity of pneumonia in a human subject comprises administering an anti-alpha toxin antibody or antigen-binding fragment thereof to the subject, wherein the antibody or antigen-binding fragment thereof comprises a set CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6, wherein the administration results in a serum level of the antibody or antigen-binding fragment (e.g., MEDI4893) of at least 211 μg/ml.

In some embodiments of the methods provided herein, the pneumonia is nosocomial pneumonia. In some embodiments, the pneumonia is Staphylococcus aureus (S. aureus) pneumonia. In some embodiments, the pneumonia is ventilator associated pneumonia. In some embodiments, the pneumonia is pneumonia following extubation.

In some embodiments, a method of decreasing nasal or tracheal colonization of S. aureus in a subject comprises administering 2000 to 5000 milligrams of an anti-alpha toxin antibody or antigen-binding fragment thereof to the subject, wherein the antibody or antigen-binding fragment thereof comprises a set of Complementarity-Determining Regions (CDRs): HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6.

In some embodiments, a method of decreasing nasal or tracheal colonization of S. aureus in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6, wherein the serum target level of the antibody or antigen-binding fragment thereof (e.g., MEDI4893) is at least 211 μg/ml.

In some embodiments, a method of decreasing nasal or tracheal colonization of S. aureus in a human subject comprises administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein, HCDR1 has the amino acid sequence of SEQ. ID. NO: 1; HCDR2 has the amino acid sequence of SEQ. ID. NO: 2; HCDR3 has the amino acid sequence of SEQ. ID. NO: 3; LCDR1 has the amino acid sequence of SEQ. ID. NO: 4; LCDR2 has the amino acid sequence of SEQ. ID. NO: 5; and LCDR3 has the amino acid sequence of SEQ. ID. NO: 6, wherein the administration results in a serum level of the antibody or antigen-binding fragment of at least 211 μg/ml.

In some embodiments, the S. aureus is resistant to methicillin. In some embodiments, the S. aureus is glycopeptide intermediately resistant. In some embodiments, the S. aureus is glycopeptide-resistant.

In some embodiments, the subject is mechanically ventilated. In some embodiments, the administration reduces the risk of pneumonia while on mechanical ventilation. In some embodiments, the administration reduces the risk of pneumonia after mechanical ventilation is no longer required.

In some embodiments, the subject is in an intensive care unit (ICU).

In some embodiments, the subject is colonized with S. aureus. In some embodiments, the subject is colonized with S. aureus in the lower respiratory tract. In some embodiments, S. aureus is present in bronchial aspirate of the subject. In some embodiments, S. aureus is present in a tracheal sample of the subject. In some embodiments, the subject is free of S. aureus-related disease.

In some embodiments, the antibody or antigen-binding fragment thereof (e.g., MEDI4893) is administered intravenously.

In some embodiments, 2000 mg of the antibody or antigen-binding fragment thereof is administered. In some embodiments, 2500 mg of the antibody or antigen-binding fragment thereof is administered. In some embodiments, 3000 mg of the antibody or antigen-binding fragment thereof is administered. In some embodiments, 3500 mg of the antibody or antigen-binding fragment thereof is administered. In some embodiments, 4000 mg of the antibody or antigen-binding fragment thereof is administered. In some embodiments, 4500 mg of the antibody or antigen-binding fragment thereof is administered. In some embodiments, 5000 mg of the antibody or antigen-binding fragment thereof is administered.

In some embodiments, the serum target level of the antibody or antigen-binding fragment thereof (e.g., MEDI4893) is at least 211 μg/ml. In some embodiments, the administration produces a serum level of the antibody or antigen-binding fragment thereof (e.g., MEDI4893) of at least 211 μg/ml.

In some embodiments, the subject received an antibiotic prior to administration of the anti-alpha toxin antibody or antigen-binding fragment thereof. In some embodiments, the anti-alpha toxin antibody or antigen-binding fragment thereof (e.g., MEDI4893) is co-administered with an antibiotic. In some embodiments, the antibiotic is vancomycin. In some embodiments, the anti-alpha toxin antibody or antigen-binding fragment thereof (e.g., MEDI4893) and the antibiotic are administered sequentially. In some embodiments, the anti-alpha toxin antibody or antigen-binding fragment thereof (e.g., MEDI4893) and the antibiotic are administered simultaneously. In some embodiments, the anti-alpha toxin antibody or antigen-binding fragment thereof (e.g., MEDI4893) and the antibiotic are administered in the same pharmaceutical composition.

In some embodiments, the anti-alpha toxin antibody or antigen-binding fragment thereof comprises a heavy chain variable region (VH) comprising SEQ ID NO:7 and a light chain variable region (VL) comprising SEQ ID NO:8.

In some embodiments, the anti-alpha toxin antibody or antigen-binding fragment thereof comprises a YTE substitution in the constant region.

In some embodiments, the anti-alpha toxin antibody or antigen-binding fragment thereof was produced by a Chinese hamster ovary (CHO) cell line.

In some embodiments, the anti-alpha toxin antibody is MEDI4893.

In some embodiments, the subject is human.

### BRIEF DESCRIPTION OF THE DRAWINGS/FIGURES

FIG. 1 provides a flow diagram of a study evaluating the efficacy and safety of the anti-alpha toxin antibody MEDI4893.

### DETAILED DESCRIPTION OF THE INVENTION

The present disclosure provides methods of preventing and/or treating a condition associated with a bacterial infection by administering an anti-alpha toxin antibody or antigen-binding fragment thereof.

In order that the present disclosure can be more readily understood, certain terms are first defined. Additional definitions are set forth throughout the detailed description.

### I. Definitions

The terms “a,” “an,” and “the” include plural referents unless the context clearly dictates otherwise. For example, “an antigen binding protein” is understood to represent one or more antigen binding proteins. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “comprise” is generally used in the sense of include, that is to say permitting the presence of one or more features or components. Wherever aspects are described herein with the language “comprising,” otherwise analogous aspects described in terms of “consisting of,” and/or “consisting essentially of” are also provided.

The term “about” as used in connection with a numerical value throughout the specification and the claims denotes an interval of accuracy, familiar and acceptable to a person skilled in the art. In general, such interval of accuracy is ±10%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

The term “antigen binding protein” refers to a molecule comprised of one or more polypeptides that recognizes and specifically binds to a target, e.g., alpha-toxin, such as an anti-alpha-toxin antibody or antigen-binding fragment thereof.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment” or “antigen-binding fragment” of an antibody refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, scFvs, and single chain antibodies.

It is possible to take monoclonal and other antibodies or fragments thereof and use techniques of recombinant DNA technology to produce other antibodies or chimeric molecules or fragments thereof that retain the specificity of the original antibody or fragment. Such techniques can involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A, or EP-A-239400, and a large body of subsequent literature. A hybridoma or other cell producing an antibody can be subject to genetic mutation or other changes, which may or may not alter the binding specificity of antibodies or fragments thereof produced.

Further techniques available in the art of antibody engineering have made it possible to isolate human and humanized antibodies or fragments thereof. For example, human hybridomas can be made as described by Kontermann and Sefan. Antibody Engineering, Springer Laboratory Manuals (2001). Phage display, another established technique for generating antigen binding proteins has been described in detail in many publications such as Kontermann and Sefan. Antibody Engineering, Springer Laboratory Manuals (2001) and WO92/01047. Transgenic mice in which the mouse antibody genes are inactivated and functionally replaced with human antibody genes while leaving intact other components of the mouse immune system, can be used for isolating human antibodies to human antigens.

Synthetic antibody molecules or fragments thereof can be created by expression from genes generated by means of oligonucleotides synthesized and assembled within suitable expression vectors, for example as described by Knappik et al. J. Mol. Biol. (2000) 296, 57-86 or Krebs et al. Journal of Immunological Methods 254 2001 67-84.

It has been shown that fragments of a whole antibody can perform the function of binding antigens. Examples of binding fragments are (i) the Fab fragment consisting of VL, VH, CL, and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989), McCafferty et al (1990) Nature, 348, 552-554) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies,” multivalent or multispecific fragments constructed by gene fusion (WO94/13804; P. Holliger et al, Proc. Natl. Acad. Sci. USA 90 6444-6448, 1993). Fv, scFv or diabody molecules may be stabilized by the incorporation of disulphide bridges linking the VH and VL domains (Y. Reiter et al, Nature Biotech, 14, 1239-1245, 1996). Minibodies comprising a scFv joined to a CH3 domain may also be made (S. Hu et al, Cancer Res., 56, 3055-3061, 1996).

The phrase “effector function” refers to the activities of antibodies that result from the interactions of their Fc components with Fc receptors or components of complement. These activities include, for example, antibody-dependent cell-mediated cytotoxicity (ADCC), complement-dependent cytotoxicity (CDC), and antibody-dependent cell phagocytosis (ADCP). Thus an antigen binding protein (e.g., an antibody or antigen binding fragment thereof) with altered effector function refers to an antigen binding protein (e.g., an antibody or antigen binding fragment thereof) that contains an alteration in an Fc region (e.g., amino acid substitution, deletion, or addition or change in oligosaccharide) that changes the activity of at least one effector function (e.g., ADCC, CDC, and/or ADCP). An antigen binding protein (e.g., an antibody or antigen binding fragment thereof) with improved effector function refers to an antigen binding protein (e.g., an antibody or antigen binding fragment thereof) that contains an alteration in an Fc region (e.g., amino acid substitution, deletion, or addition or change in oligosaccharide) that increases the activity of at least one effector function (e.g., ADCC, CDC, and/or ADCP).

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the antigen binding protein carrying the antigen binding domain will be able to bind to the various antigens carrying the epitope.

By “specifically binds” it is generally meant that an antigen binding protein including an antibody or antigen binding fragment thereof binds to an epitope via its antigen binding domain, and that the binding entails some complementarity between the antigen binding domain and the epitope. According to this definition, an antibody is said to “specifically bind” to an epitope when it binds to that epitope via its antigen binding domain more readily than it would bind to a random, unrelated epitope.

“Affinity” is a measure of the intrinsic binding strength of a ligand binding reaction. For example, a measure of the strength of the antibody (Ab)-antigen (Ag) interaction is measured through the binding affinity, which may be quantified by the dissociation constant, k_{d}. The dissociation constant is the binding affinity constant and is given by:

$${K}_{d} = \frac{[ Ab ] [ Ag ]}{[ AbAg complex ]}$$

Affinity may, for example, be measured using a BIAcore®, a KinExA affinity assay, flow cytometry, and/or radioimmunoassay.

“Potency” is a measure of pharmacological activity of a compound expressed in terms of the amount of the compound required to produce an effect of given intensity. It refers to the amount of the compound required to achieve a defined biological effect; the smaller the dose required, the more potent the drug. Potency of an antigen binding protein that binds alpha toxin may, for example, be determined using an OPK assay, as described herein.

An antigen binding protein including an antibody or antigen binding fragment thereof is said to competitively inhibit binding of a reference antibody or antigen binding fragment thereof to a given epitope or “compete” with a reference antibody or antigen binding fragment if it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope or compete with a reference antibody or antigen binding fragment thereof by at least 90%, at least 80%, at least 70%, at least 60%, or at least 50%.

The term “compete” when used in the context of antigen binding proteins (e.g., neutralizing antigen binding proteins or neutralizing antibodies) means competition between antigen binding proteins as determined by an assay in which the antigen binding protein (e.g., antibody or immunologically functional fragment thereof) under test prevents or inhibits specific binding of a reference antigen binding protein (e.g., a ligand, or a reference antibody) to a common antigen (e.g., an alpha toxin protein or a fragment thereof). Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 92:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabeled test antigen binding protein and a labeled reference antigen binding protein.

Competitive inhibition can be measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Usually, when a competing antigen binding protein is present in excess, it will inhibit specific binding of a reference antigen binding protein to a common antigen by at least 40%, 45%, 50%, 55%, 60%, 65%, 70% or 75%. In some instance, binding is inhibited by at least 80%, 85%, 90%, 91%, 92%, 93%, 94%, 95%, 96%, 97% 98%, 99% or more.

Antigen binding proteins, antibodies or antigen binding fragments thereof disclosed herein can be described or specified in terms of the epitope(s) or portion(s) of an antigen, e.g., a target polypeptide that they recognize or specifically bind. For example, the portion of alpha toxin that specifically interacts with the antigen binding domain of the antigen binding polypeptide or fragment thereof disclosed herein is an “epitope”. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. A conformational epitope can be composed of discontinuous sections of the antigen's amino acid sequence. A linear epitope is formed by a continuous sequence of amino acids from the antigen. Epitope determinants may include chemically active surface groupings of molecules such as amino acids, sugar side chains, phosphoryl or sulfonyl groups, and can have specific three dimensional structural characteristics, and/or specific charge characteristics. An epitope typically includes at least 3, 4, 5, 6, 7, 8, 9, 10, 11, 12, 13, 14, 15, 16, 17, 18, 19, 20, 25, 30, or 35 amino acids in a unique spatial conformation. Epitopes can be determined using methods known in the art.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The term “isolated” refers to the state in which antigen binding proteins of the disclosure, or nucleic acid encoding such binding proteins, will generally be in accordance with the present disclosure. Isolated proteins and isolated nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practiced in vitro or in vivo. Proteins and nucleic acid may be formulated with diluents or adjuvants and still for practical purposes be isolated—for example the proteins will normally be mixed with gelatin or other carriers if used to coat microtitre plates for use in immunoassays, or will be mixed with pharmaceutically acceptable carriers or diluents when used in diagnosis or therapy. Antigen binding proteins may be glycosylated, either naturally or by systems of heterologous eukaryotic cells (e.g. CHO or NS0 (ECACC 85110503) cells, or they may be (for example if produced by expression in a prokaryotic cell) unglycosylated.

A polypeptide, antigen binding protein, antibody, polynucleotide, vector, cell, or composition which is “isolated” is a polypeptide, antigen binding protein, antibody, polynucleotide, vector, cell, or composition which is in a form not found in nature. Isolated polypeptides, antigen binding proteins, antibodies, polynucleotides, vectors, cells, or compositions include those which have been purified to a degree that they are no longer in a form in which they are found in nature. In some embodiments, an antigen binding protein, antibody, polynucleotide, vector, cell, or composition which is isolated is substantially pure.

A “recombinant” polypeptide, protein or antibody refers to a polypeptide or protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

Also included in the present disclosure are fragments, variants, or derivatives of polypeptides, and any combination thereof. The term “fragment” when referring to polypeptides and proteins of the present disclosure include any polypeptides or proteins which retain at least some of the properties of the reference polypeptide or protein. Fragments of polypeptides include proteolytic fragments, as well as deletion fragments.

The term “variant” as used herein refers to an antibody or polypeptide sequence that differs from that of a parent antibody or polypeptide sequence by virtue of at least one amino acid modification. Variants of antibodies or polypeptides of the present disclosure include fragments, and also antibodies or polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “derivatives” as applied to antibodies or polypeptides refers to antibodies or polypeptides which have been altered so as to exhibit additional features not found on the native polypeptide or protein. An example of a “derivative” antibody is a fusion or a conjugate with a second polypeptide or another molecule (e.g., a polymer such as PEG, a chromophore, or a fluorophore) or atom (e.g., a radioisotope).

The terms “polynucleotide” or “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “host cell” refers to a cell or a population of cells harboring or capable of harboring a recombinant nucleic acid. Host cells can be prokaryotic cells (e.g., E. coli), or alternatively, the host cells can be eukaryotic, for example, fungal cells (e.g., yeast cells such as Saccharomyces cerivisiae, Pichia pastoris, or Schizosaccharomyces pombe), and various animal cells, such as insect cells (e.g., Sf-9) or mammalian cells (e.g., HEK293F, CHO, COS-7, NIH-3T3, a NS0 murine myeloma cell, a PER.C6® human cell, a Chinese hamster ovary (CHO) cell or a hybridoma).

The term “amino acid substitution” refers to replacing an amino acid residue present in a parent sequence with another amino acid residue. An amino acid can be substituted in a parent sequence, for example, via chemical peptide synthesis or through recombinant methods known in the art. Accordingly, references to a “substitution at position X” or “substitution at position X” refer to the substitution of an amino acid present at position X with an alternative amino acid residue. In some embodiments, substitution patterns can be described according to the schema AXY, wherein A is the single letter code corresponding to the amino acid naturally present at position X, and Y is the substituting amino acid residue. In other aspects, substitution patterns can described according to the schema XY, wherein Y is the single letter code corresponding to the amino acid residue substituting the amino acid naturally present at position X.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

Non-conservative substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala, Ser) or no side chain (e.g., Gly).

Other substitutions can be readily identified by workers of ordinary skill. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, omithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

The term “amino acid insertion” refers to introducing a new amino acid residue between two amino acid residues present in the parent sequence. An amino acid can be inserted in a parent sequence, for example, via chemical peptide synthesis or through recombinant methods known in the art. Accordingly as used herein, the phrases “insertion between positions X and Y” or “insertion between Kabat positions X and Y,” wherein X and Y correspond to amino acid positions (e.g., a cysteine amino acid insertion between positions 239 and 240), refers to the insertion of an amino acid between the X and Y positions, and also to the insertion in a nucleic acid sequence of a codon encoding an amino acid between the codons encoding the amino acids at positions X and Y. Insertion patterns can be described according to the schema AXins, wherein A is the single letter code corresponding to the amino acid being inserted, and X is the position preceding the insertion.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

“Specific binding member” describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. The present disclosure is concerned with antigen-antibody type reactions.

The term “IgG” as used herein refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3.

The term “monoclonal” refers to a homogeneous antibody population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” refers to such antibodies and antigen-binding fragments thereof made in any number of ways including, but not limited to, by hybridoma, phage selection, recombinant expression, and transgenic animals.

The term “human” antibody or antigen-binding fragment thereof refers to an antibody or antigen-binding fragment thereof produced by a human or having an amino acid sequence corresponding to an antibody produced by a human made using any technique known in the art. This definition of a human antibody or antigen-binding fragment thereof includes intact or full-length antibodies, fragments thereof, and/or antibodies comprising at least one human heavy and/or light chain polypeptide such as, for example, an antibody comprising murine light chain and human heavy chain polypeptides. The term “humanized” refers to an antibody or antigen-binding fragment thereof derived from a non-human (e.g., murine) immunoglobulin, which has been engineered to contain minimal non-human (e.g., murine) sequences.

The term “chimeric” refers to antibodies or antigen-binding fragments thereof wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The term “antibody binding site” refers to a region in the antigen (e.g., alpha toxin) comprising a continuous or discontinuous site (i.e., an epitope) to which a complementary antibody specifically binds. Thus, the antibody binding site can contain additional areas in the antigen which are beyond the epitope and which can determine properties such as binding affinity and/or stability, or affect properties such as antigen enzymatic activity or dimerization. Accordingly, even if two antibodies bind to the same epitope within an antigen, if the antibody molecules establish distinct intermolecular contacts with amino acids outside of the epitope, such antibodies are considered to bind to distinct antibody binding sites.

The Kabat numbering system is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The phrases “amino acid position numbering as in Kabat,” “Kabat position,” and grammatical variants thereof refer to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The IMGT (Lefranc, M.-P. et al. Dev. Comp. Immunol. 27: 55-77 (2003)) classification of CDRs can also be used.

The term “EU index as in Kabat” refers to the numbering system of the human IgG1 EU antibody described in Kabat et al., Sequences of Immunological Interest, 5th Ed. Public Health Service, National Institutes of Health, Bethesda, Md. (1991). All amino acid positions referenced in the present application refer to EU index positions. For example, both “L234” and “EU L234” refer to the amino acid leucine at position 234 according to the EU index as set forth in Kabat.

The terms “Fc domain,” “Fc Region,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor. For example, an Fc domain contains the entire second constant domain CH2 (residues at EU positions 231-340 of human IgG1) and the third constant domain CH3 (residues at EU positions 341-447 of human IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358. Thus, a “wild type IgG Fc domain” or “WT IgG Fc domain” refers to any naturally occurring IgG Fc region (i.e., any allele). Myriad Fc mutants, Fc fragments, Fc variants, and Fc derivatives are described, e.g., in U.S. Pat. Nos. 5,624,821; 5,885,573; 5,677,425; 6,165,745; 6,277,375; 5,869,046, 6,121,022, 5,624,821, 5,648,260, 6,528,624, 6,194,551, 6,737,056, 7,122,637, 7,183,387; 7,332,581; 7,335,742; 7,371,826; 6,821,505; 6,180,377; 7,317,091; 7,355,008; U.S. Patent publication 2004/0002587; and PCT Publication Nos. WO 99/058572, WO 2011/069164 and WO 2012/006635.

The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “YTE” or “YTE mutant” refer to a set of mutations in an IgG1 Fc domain that results in an increase in the binding to human FcRn and improves the serum half-life of the antibody having the mutation. A YTE mutant comprises a combination of three “YTE mutations”: M252Y, S254T, and T256E, wherein the numbering is according to the EU index as in Kabat, introduced into the heavy chain of an IgG. See U.S. Pat. No. 7,658,921, which is incorporated by reference herein. The YTE mutant has been shown to increase the serum half-life of antibodies compared to wild-type versions of the same antibody. See, e.g., Dall'Acqua et al., J. Biol. Chem. 281:23514-24 (2006) and U.S. Pat. No. 7,083,784, which are hereby incorporated by reference in their entireties. A “Y” mutant comprises only the M256Y mutations; similarly a “YT” mutation comprises only the M252Y and S254T; and a “YE” mutation comprises only the M252Y and T256E. It is specifically contemplated that other mutations may be present at EU positions 252 and/or 256. In certain aspects, the mutation at EU position 252 may be M252F, M252S, M252W or M252T and/or the mutation at EU position 256 may be T256S, T256R, T256Q or T256D.

The term “naturally occurring alpha toxin” generally refers to a state in which the alpha toxin protein or fragments thereof can occur. Naturally occurring alpha toxin means alpha toxin protein which is naturally produced by a cell, without prior introduction of encoding nucleic acid using recombinant technology. Thus, naturally occurring alpha toxin can be as produced naturally by for example Staphylococcus aureus and/or as isolated from different members of the Staphylococcus genus.

The term “recombinant alpha toxin” refers to a state in which the alpha toxin protein or fragments thereof can occur. Recombinant alpha toxin means alpha toxin protein or fragments thereof produced by recombinant DNA, e.g., in a heterologous host. Recombinant alpha toxin can differ from naturally occurring alpha toxin by glycosylation pattern.

Recombinant proteins expressed in prokaryotic bacterial expression systems are not glycosylated while those expressed in eukaryotic systems such as mammalian or insect cells are glycosylated. Proteins expressed in insect cells however differ in glycosylation from proteins expressed in mammalian cells.

The terms “half-life” or “in vivo half-life” as used herein refer to the biological half-life of a particular type of antibody, antigen binding protein, or polypeptide of the present disclosure in the circulation of a given animal and is represented by a time required for half the quantity administered in the animal to be cleared from the circulation and/or other tissues in the animal.

The term “subject” as used herein refers to any animal (e.g., a mammal), including, but not limited to humans, non-human primates, rodents, sheep, dogs, cats, horses, cows, bears, chickens, amphibians, reptiles, and the like, which is to be the recipient of a particular treatment. The terms “subject” and “patient” as used herein refer to any subject, particularly a mammalian subject, for whom diagnosis, prognosis, or therapy of a condition associated with a bacterial, e.g., Staphylococcus infection. As used herein, phrases such as “a patient having a condition associated with a Staphylococcus infection” includes subjects, such as mammalian subjects, that would benefit from the administration of a therapy, imaging or other diagnostic procedure, and/or preventive treatment for that condition associated with a Staphylococcus infection.

The term “pharmaceutical composition” as used herein refers to a preparation which is in such form as to permit the biological activity of the active ingredient to be effective, and which contains no additional components which are unacceptably toxic to a subject to which the composition would be administered. Such composition can be sterile.

The term “label” when used herein refers to a detectable compound or composition which is conjugated directly or indirectly to a polypeptide, e.g., an antigen binding protein including an antibody, so as to generate a “labeled” polypeptide or antibody. The label can be detectable by itself (e.g., radioisotope labels or fluorescent labels) or, in the case of an enzymatic label, can catalyze chemical alteration of a substrate compound or composition which is detectable.

Terms such as “treating” or “treatment” or “to treat” refer to therapeutic measures that cure and/or halt progression of a diagnosed pathologic condition or disorder. Terms such as “preventing” refer to prophylactic or preventative measures that prevent and/or slow the development of a targeted pathologic condition or disorder (e.g., a nosocomial infection or pneumonia). Thus, those in need of treatment include those already with the disease or condition. Those in need of prevention include those prone to have the disease or condition and those in whom the disease or condition is to be prevented. For example, the phrase “treating a patient” having a bacteria-mediated disease or condition (e.g., a nosocomial infection or pneumonia), refers to reducing the severity of the bacteria-mediated disease or condition to an extent that the subject no longer suffers discomfort and/or altered function due to it. The phrase “preventing” a bacteria-mediated disease or condition (e.g., a nosocomial infection or pneumonia), refers to reducing the potential for a bacteria-mediated disease or condition and/or reducing the occurrence of the bacteria-mediated disease or condition (for example a relative reduction in occurrence as compared to untreated patients).

Terms such as “decreasing the severity” refer to therapeutic measures that slow down or lessen the symptoms of a diagnosed pathologic condition or disorder. For example, the phrase “decreasing the severity” of a bacteria-mediated disease or condition (e.g., a nosocomial infection or pneumonia) refers to reducing the severity of the bacteria-mediated disease or condition (for example, a relative reduction in symptoms when compared to untreated patients). Decreasing the severity of a bacteria-mediated disease or condition (e.g., a nosocomial infection or pneumonia) can include, for example, reduced length of hospital stay, reduced length of ICU stay, reduced time of antibiotic usage, reduced time of mechanical ventilation, decreased clinical pulmonary infection score, and/or decreased Sequential Organ Failure Assessment (SOFA) score, and/or increased EQ-5D-5L Health Questionnaire score.

As used herein, the term “a condition associated with an S. aureus infection” refers to any pathology caused by (alone or in association with other mediators), exacerbated by, associated with, or prolonged by S. aureus infection in the subject having the disease or condition. Non-limiting examples of conditions associated with an S. aureus infection include pneumonia, bacteremia, sepsis, bone and joint infections, deep skin or tissue infection, meningitis, and endocarditis. In some embodiments, the S. aureus infection is a nosocomial infection. In some embodiments, the S. aureus infection is an opportunistic infection. In some embodiments, the S. aureus infection follows an organ transplant. In some embodiments, the subject is exposed to a S. aureus contaminated medical device, including, e.g., a ventilator, a surgical tool, a catheter, or an intravenous catheter.

The structure for carrying a CDR or a set of CDRs will generally be of an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains may be determined by reference to Kabat, E. A. et al, Sequences of Proteins of Immunological Interest. 4th Edition. US Department of Health and Human Services. 1987, and updates thereof, now available on the Internet (http://immuno.bme.nwu.edu or find “Kabat” using any search engine), herein incorporated by reference. CDRs can also be carried by other scaffolds such as fibronectin or cytochrome B.

A CDR amino acid sequence substantially as set out herein can be carried as a CDR in a human variable domain or a substantial portion thereof. The HCDR3 sequences substantially as set out herein represent embodiments of the present disclosure and each of these may be carried as a HCDR3 in a human heavy chain variable domain or a substantial portion thereof.

Variable domains employed in the disclosure can be obtained from any germ-line or rearranged human variable domain, or can be a synthetic variable domain based on consensus sequences of known human variable domains. A CDR sequence (e.g. CDR3) can be introduced into a repertoire of variable domains lacking a CDR (e.g. CDR3), using recombinant DNA technology.

For example, Marks et al. (Bio/Technology, 1992, 10:779-783; which is incorporated herein by reference) provide methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire can be combined with a CDR3 of a particular antibody. Using analogous techniques, the CDR3-derived sequences of the present disclosure can be shuffled with repertoires of VH or VL domains lacking a CDR3, and the shuffled complete VH or VL domains combined with a cognate VL or VH domain to provide antigen binding proteins. The repertoire can then be displayed in a suitable host system such as the phage display system of WO92/01047 or any of a subsequent large body of literature, including Kay, B. K., Winter, J., and McCafferty, J. (1996) Phage Display of Peptides and Proteins: A Laboratory Manual, San Diego: Academic Press, so that suitable antigen binding proteins may be selected. A repertoire can consist of from anything from 104 individual members upwards, for example from 106 to 108 or 110 members. Other suitable host systems include yeast display, bacterial display, T7 display, ribosome display and so on. For a review of ribosome display see Lowe D and Jermutus L, 2004, Curr. Pharm, Biotech, 517-27, also WO92/01047, which are herein incorporated by reference.

Analogous shuffling or combinatorial techniques are also disclosed by Stemmer (Nature, 1994, 370:389-391, which is herein incorporated by reference), who describes the technique in relation to a β-lactamase gene but observes that the approach may be used for the generation of antibodies.

A further alternative is to generate novel VH or VL regions carrying CDR-derived sequences of the disclosure using random mutagenesis of one or more selected VH and/or VL genes to generate mutations within the entire variable domain. Such a technique is described by Gram et al (1992, Proc. Natl. Acad. Sci., USA, 89:3576-3580), who used error-prone PCR. In some embodiments, one or two amino acid substitutions are made within a set of HCDRs and/or LCDRs.

Another method which may be used is to direct mutagenesis to CDR regions of VH or VL genes. Such techniques are disclosed by Barbas et al, (1994, Proc. Natl. Acad. Sci., USA, 91:3809-3813) and Schier et al (1996, J. Mol. Biol. 263:551-567).

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), all of which are herein incorporated by reference.

### II. Alpha Toxin Binding Molecules

In certain aspects, the antibody or antigen-binding fragment thereof for use according to the methods provided herein specifically binds to a Staphylococcus aureus (S. aureus) alpha toxin. Alpha toxin (also known as alpha-hemolysin or Hla) is a pore-forming and hemolytic exoprotein produced by most pathogenic strains of S. aureus. The toxin forms heptameric pores in membranes of susceptible cells such as white blood cells, platelets, erythrocytes, peripheral blood monocytes, macrophages, keratinocytes, fibroblasts and endothelial cells. Alpha toxin pore formation often leads to cell dysfunction or lysis. In certain aspects, the S. aureus alpha toxin comprises the amino acid sequence of SEQ ID NO: 11.

(SEQ ID NO: 11)
ADSDINIKTGTTDIGSNTTVKTGDLVTYDKENGMHKKVFYSFIDDKNHNK
KLLVIRTKGTIAGQYRVYSEEGANKSGLAWPSAFKVQLQLPDNEVAQISD
YYPRNSIDTKEYMSTLTYGFNGNVTGDDTGKIGGLIGANVSIGHTLKYVQ
PDFKTILESPTDKKVGWKVIFNNMVNQNWGPYDRDSWNPVYGNQLFMKTR
NGSMKAADNFLDPNKASSLLSSGFSPDFATVITMDRKASKQQTNIDVIYE
RVRDDYQLHWTSTNWKGTNTKDKWTDRSSERYKIDWEKEEMTN

In certain aspects, the antibody or antigen-binding fragment thereof that specifically binds to S. aureus alpha toxin comprises the CDR, VH, and/or VL sequences of an antibody or an antigen-binding fragment thereof disclosed in WO 2012/109285 (which is herein incorporated by reference in its entirety), e.g., the LC10 antibody.

In certain aspects, the antibody or antigen-binding fragment thereof that specifically binds to S. aureus alpha toxin comprises a set of complementarity-determining regions (CDRs): HCDR1, HCDR2, HCDR3, LCDR1, LCDR2, and LCDR3, wherein HCDR1 has the amino acid sequence of SEQ ID NO:1 (SHDMH), HCDR2 has the amino acid sequence of SEQ ID NO:2 (GIGTAGDTYYPDSVKG), HCDR3 has the amino acid sequence of SEQ ID NO:3 (DRYSPTGHYYGMDV), LCDR1 has the amino acid sequence of SEQ ID NO:4 (RASQSISSWLA), LCDR2 has the amino acid sequence of SEQ ID NO:5 (KASSLES), and LCDR3 has the amino acid sequence of SEQ ID NO:6 (KQYADYWT).

In certain aspects, the antibody or antigen-binding fragment thereof that specifically binds to S. aureus alpha toxin comprises a variable heavy chain comprising an amino acid sequence at least 90% identical to the amino acid sequence of SEQ ID NO:7 and a variable light chain comprising an amino acid sequence at least 90% identical to the amino acid sequence of SEQ ID NO:8.

(SEQ ID NO: 7)
EVQLVESGGGLVQPGGSLRLSCAASGFTFSSHDMHWVRQATGKGLEWVSG
IGTAGDTYYPDSVKGRFTISRENAKNSINLQMNSLRAGDTAVYYGARDRY
SPTGHYYGMDVWGQGTTVTVSS
(SEQ ID NO: 8)
DIQMTQSPSTLSASVGDRVTITCRASQSISSWLAWYQQKPGKAPKLLIYK
ASSLESGVPSRFSGSGSGTEFTLTISSLQPDDFATYYCKQYADYWTFGQG
TKVEIK

In certain aspects, the antibody or antigen-binding fragment thereof that specifically binds to S. aureus alpha toxin comprises a variable heavy chain comprising an amino acid sequence at least 95% identical to the amino acid sequence of SEQ ID NO:7 and a variable light chain comprising an amino acid sequence at least 95% identical to the amino acid sequence of SEQ ID NO:8.

In certain aspects, the antibody or antigen-binding fragment thereof that specifically binds to S. aureus alpha toxin comprises a variable heavy chain comprising an amino acid sequence at least 96% identical to the amino acid sequence of SEQ ID NO:7 and a variable light chain comprising an amino acid sequence at least 96% identical to the amino acid sequence of SEQ ID NO:8.

In certain aspects, the antibody or antigen-binding fragment thereof that specifically binds to S. aureus alpha toxin comprises a variable heavy chain comprising an amino acid sequence at least 97% identical to the amino acid sequence of SEQ ID NO:7 and a variable light chain comprising an amino acid sequence at least 97% identical to the amino acid sequence of SEQ ID NO:8.

In certain aspects, the antibody or antigen-binding fragment thereof that specifically binds to S. aureus alpha toxin comprises a variable heavy chain comprising an amino acid sequence at least 98% identical to the amino acid sequence of SEQ ID NO:7 and a variable light chain comprising an amino acid sequence at least 98% identical to the amino acid sequence of SEQ ID NO:8.

In certain aspects, the antibody or antigen-binding fragment thereof that specifically binds to S. aureus alpha toxin comprises a variable heavy chain comprising an amino acid sequence at least 99% identical to the amino acid sequence of SEQ ID NO:7 and a variable light chain comprising an amino acid sequence at least 99% identical to the amino acid sequence of SEQ ID NO:8.

In certain aspects, the antibody or antigen-binding fragment thereof that specifically binds to S. aureus alpha toxin comprises a variable heavy chain comprising the amino acid sequence of SEQ ID NO:7 and a variable light chain comprising the amino acid sequence of SEQ ID NO:8.

In certain aspects, the antibody or antigen-binding fragment thereof that specifically binds to S. aureus alpha toxin comprises a heavy chain comprising the amino acid sequence of SEQ ID NO:9 and a light chain comprising the amino acid sequence of SEQ ID NO:10 (i.e., MEDI4893).

(SEQ ID NO: 9)
EVQLVESGGGLVQPGGSLRLSCAASGFTFSSHDMHWVRQATGKGLEWVSG
IGTAGDTYYPDSVKGRFTISRENAKNSLYLQMNSLRAGDTAVYYCARDRY
SPTGHYYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
(SEQ ID NO: 10)
DIQMTQSPSTLSASVGDRVTITCRASQSISSWLAWYQQKPGKAPKLLIYK
ASSLESGVPSRFSGSGSGTEFTLTISSLQPDDFATYYCKQYADYWTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGE

In certain aspects, the antibody or antigen-binding fragment thereof that specifically binds to S. aureus alpha toxin, binds to the same epitope as an antibody comprising comprises a heavy chain comprising the amino acid sequence of SEQ ID NO:9 and a light chain comprising the amino acid sequence of SEQ ID NO: 10.

In certain aspects, the antibody or antigen-binding fragment thereof that specifically binds to S. aureus alpha toxin, competitively inhibits the binding of an antibody comprising a heavy chain comprising the amino acid sequence of SEQ ID NO:9 and a light chain comprising the amino acid sequence of SEQ ID NO: 10 to S. aureus alpha toxin.

In certain aspects, the antibody or antigen-binding fragment thereof that specifically binds to S. aureus alpha toxin (a) has an affinity constant (K_{D}) for alpha toxin of about 13 nM or less; (b) binds to alpha toxin monomers, but does not inhibit binding of alpha toxin to alpha toxin receptor; (c) inhibits the formation of alpha toxin oligomers by at least 50%, 60%, 70%, 80%, 90%, or 95%; (d) reduces alpha toxin cytolytic activity by at least 50%, 60%, 70%, 80%, 90%, or 95% (e.g., as determined by cell lysis and hemolysis assays); (e) reduces cell infiltration and pro-inflammatory cytokine release; or (f) a combination thereof. In certain aspects, the antibody or antigen-binding fragment thereof that specifically binds to S. aureus alpha toxin blocks alpha toxin pore formation in cell membranes.

In certain embodiments, an antibody or antigen-binding fragment thereof as described herein specifically binds to an S. aureus epitope (e.g., alpha toxin) with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In certain embodiments, an anti-alpha toxin antibody or antigen-binding fragment alters the biological properties of alpha toxin, alpha toxin expressing cells, or other bacterial cells. In certain aspects, an anti-alpha toxin antibody or antigen-binding fragment neutralizes the biological activity of alpha toxin by binding to the polypeptide and inhibiting the assembly of alpha toxin monomers into a transmembrane pore (e.g., alpha toxin heptamer). Neutralization assays can be performed using methods known in the art using, in some circumstances, commercially available reagents. Neutralization of alpha toxin often is measured with an IC50 of 1×10⁻⁶ M or less, 1×10⁻⁷ M or less, 1×10⁻⁸ M or less, 1×10⁻⁹ M or less, 1×10⁻¹⁰ M or less and 1×10⁻¹¹ M or less. In certain embodiments, an anti-alpha toxin antibody or fragment neutralizes the ability of alpha toxin to oligomerize and form a transmembrane pore. The term “inhibitory concentration 50%” (abbreviated as “IC50”) represents the concentration of an inhibitor (e.g., an anti-alpha toxin antibody or fragment provided herein) that is required for 50% inhibition of a given activity of the molecule the inhibitor targets (e.g., alpha toxin oligomerization to form a transmembrane pore heptamer complex). A lower IC50 value generally corresponds to a more potent inhibitor.

In certain embodiments, an anti-alpha toxin antibody or fragment inhibits one or more biological activities of alpha toxin. The term “inhibition” as used herein, refers to any statistically significant decrease in biological activity, including full blocking of the activity. For example, “inhibition” can refer to a decrease of about 10%, 20%, 30%, 40%, 50%, 60%, 70%, 80%, 90%, or 100% in biological activity. In certain embodiments, an anti-alpha toxin antibody or fragment inhibits one or more biological activities of alpha toxin by at least 10%, 20%, 30%, 40%, 50%, 60%, 70%, 80%, 90%, or 100%.

In certain aspects, an anti-alpha toxin antibody or fragment can deplete alpha toxin secreted by pathogenic S. aureus. In certain aspects, an anti-alpha toxin antibody or fragment may achieve at least about 20%, at least about 30%, at least about 40%, at least about 50%, at least about 60%, at least about 70%, at least about 80%, at least about 90%, at least about 95%, or about 100% depletion of alpha toxin secreted by S. aureus.

In certain embodiments, an anti-alpha toxin antibody or fragment can inhibit in vitro stimulated alpha toxin activity (e.g., receptor binding, oligomerization) and/or proliferation of cells expressing or secreting alpha toxin. An anti-alpha toxin antibody or fragment sometimes inhibits in vitro alpha toxin activity, S. aureus pathogenicity by at least about 10%, at least about 20%, at least about 30%, at least about 40%, at least about 50% or at least about 75%. Methods for measuring cell proliferation, pathogenicity, and alpha hemolysin activity are known in the art.

In certain embodiments, an anti-alpha toxin antibody or antigen-binding fragment can inhibit the expression of one or more inducible genes that responds directly or indirectly to the environment created by S. aureus infection and/or alpha toxin expression and function. In specific embodiments, an anti-alpha toxin antibody or antigen-binding fragment inhibits the expression of one or more inducible genes that responds directly or indirectly to the environment created by S. aureus infection and/or alpha toxin expression and function by at least 20%, by at least 300′%, by at least 40%, by at least 50%, by at least 60%, by at least 70%, by at least 80%, by at least 90%, by at least 100%, by at least 120%, by at least 140%, by at least 160%, by at least 180%/, or by at least 200%.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells. Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597). In some embodiments, the antibody or antigen-binding fragment thereof is expressed by CHO cells.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the monoclonal antibody against S. aureus alpha toxin is a humanized antibody. In certain embodiments, such antibodies are used therapeutically to reduce antigenicity and HAMA (human anti-mouse antibody) responses when administered to a human subject. Humanized antibodies can be produced using various techniques known in the art.

In some embodiments, the antibody to S. aureus alpha toxin is a human antibody. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373). Also, the human antibody can be selected from a phage library, where that phage library expresses human antibodies, as described, for example, in Vaughan et al., 1996, Nat. Biotech., 14:309-314, Sheets et al., 1998, Proc. Nat'l. Acad. Sci., 95:6157-6162, Hoogenboom and Winter, 1991, J. Mol. Biol., 227:381, and Marks et al., 1991, J. Mol. Biol., 222:581). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., 2007, J. Mol. Bio., doi:10.1016/j.jmb.2007.12.018 (each of which is incorporated by reference in its entirety). Affinity maturation strategies and chain shuffling strategies (Marks et al., 1992, Bio/Technology 10:779-783, incorporated by reference in its entirety) are known in the art and can be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

According to the present disclosure, techniques can be adapted for the production of single-chain antibodies specific to S. aureus alpha toxin (see U.S. Pat. No. 4,946,778). In addition, methods can be adapted for the construction of Fab expression libraries (Huse, et al., Science 246:1275-1281 (1989)) to allow rapid and effective identification of monoclonal Fab fragments with the desired specificity for S. aureus alpha toxin, or fragments thereof. Antibody fragments can be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antigen binding proteins of the present disclosure can further comprise antibody constant regions or parts thereof. For example, a VL domain can be attached at its C-terminal end to antibody light chain constant domains including human Cκ or Cγ chains. Similarly, an antigen binding protein based on a VH domain can be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1 and IgG4. For example, the immunoglobulin heavy chain can be derived from the antibody isotype sub-class, IgG1. Any synthetic or other constant region variant that has these properties and stabilizes variable regions is also contemplated for use in embodiments of the present disclosure.

The antibody constant region can be an Fc region with a YTE mutation, such that the Fc region comprises the following amino acid substitutions: M252Y/S254T/T256E. This residue numbering is based on Kabat numbering. The YTE mutation in the Fc region increases serum persistence of the antigen-binding protein (see Dall'Acqua, W. F. et al. (2006) The Journal of Biological Chemistry, 281, 23514-23524). The antigen-binding protein of the disclosure can comprise an IgG Fc domain containing a mutation at positions 252, 254 and 256, wherein the position numbering is according to the EU index as in Kabat. For example, the IgG1 Fc domain can contain a mutation of M252Y, S254T, and T256E, wherein the position numbering is according to the EU index as in Kabat.

In some embodiments herein, the antigen binding protein, e.g., antibody or antigen-binding fragment thereof is modified to improve effector function, e.g., so as to enhance antigen-dependent cell-mediated cytotoxicity (ADCC) and/or complement dependent cytotoxicity (CDC). This can be achieved by making one or more amino acid substitutions or by introducing cysteine in the Fc region. Variants of the Fc region (e.g., amino acid substitutions and/or additions and/or deletions) that can enhance or diminish effector function of an antibody and/or alter the pharmacokinetic properties (e.g., half-life) of the antibody are disclosed, for example in U.S. Pat. No. 6,737,056B1, U.S. Patent Application Publication No. 2004/0132101A1, U.S. Pat. Nos. 6,194,551, and 5,624,821 and 5,648,260. One particular set of substitutions, the triple mutation L234F/L235E/P331S (“TM”) causes a profound decrease in the binding activity of human IgG1 molecules to human C1q, CD64, CD32A and CD16. See, e.g., Oganesyan et al., Acta Crystallogr D Biol Crystallogr. 64:700-704 (2008). In other cases it can be that constant region modifications increase serum half-life. The serum half-life of proteins comprising Fc regions can be increased by increasing the binding affinity of the Fc region for FcRn.

When the antigen-binding protein is an antibody or an antigen-binding fragment thereof, it can further comprise a heavy chain immunoglobulin constant domain selected from the group consisting of: (a) an IgA constant domain; (b) an IgD constant domain; (c) an IgE constant domain; (d) an IgG1 constant domain; (e) an IgG2 constant domain; (f) an IgG3 constant domain; (g) an IgG4 constant domain; and (h) an IgM constant domain. In some embodiments, the antigen-binging protein is an antibody or an antigen-binding fragment thereof that comprises an IgG heavy chain immunoglobulin constant domain.

The antigen-binding protein of the disclosure can further comprise a light chain immunoglobulin constant domain selected from the group consisting of: (a) an Ig kappa constant domain; and (b) an Ig lambda constant domain. In some embodiments, the antigen-binding protein is an antibody or an antigen-binding fragment thereof that comprises a kappa constant domain.

The antigen-binding protein of the disclosure can further comprise a human IgG1 constant domain and a human kappa constant domain.

### III. Pharmaceutical Compositions

The disclosure also provides a pharmaceutical composition comprising one or more of the anti-S. aureus alpha toxin antibodies or antigen binding fragments thereof described herein. In certain embodiments, the pharmaceutical compositions further comprise a pharmaceutically acceptable vehicle or pharmaceutically acceptable excipient. In certain embodiments, these pharmaceutical compositions find use in treating, preventing or ameliorating a condition associated with a bacterial infection, e.g., an S. aureus infection, in human patients. In certain embodiments, these pharmaceutical compositions find use in inhibiting growth of S. aureus.

In certain embodiments, formulations are prepared for storage and use by combining an anti-S. aureus alpha toxin antibody or antigen binding fragment thereof described herein with a pharmaceutically acceptable vehicle (e.g., carrier, excipient) (see, e.g., Remington, The Science and Practice of Pharmacy 20th Edition Mack Publishing, 2000, herein incorporated by reference). In some embodiments, the formulation comprises a preservative.

The pharmaceutical compositions of the present disclosure can be administered in any number of ways including systemic treatment, e.g., intravenous (IV) administration. Therapeutic compositions of the present technology can be formulated for particular routes of administration. In certain embodiments, a pharmaceutical composition is formulated for intravenous (IV) administration.

In some embodiments, a pharmaceutical composition comprising one or more of the anti-S. aureus alpha toxin antibodies or antigen binding fragments thereof described herein is used for treating pneumonia, bacteremia, bone and joint infections, deep skin or tissue infection, meningitis, or endocarditis due to a bacterial infection (e.g., an S. aureus infection). In some embodiments, a pharmaceutical composition comprising one or more of the anti-S. aureus alpha toxin antibodies or antigen binding fragments thereof described herein is useful in nosocomial infections, opportunistic infections, infections following organ transplants, and other conditions associated with a bacterial infection (e.g. infection with S. aureus). In some embodiments, a pharmaceutical composition comprising one or more of the anti-S. aureus alpha toxin antibodies or antigen binding fragments thereof described herein is useful in subjects exposed to a bacterially contaminated device, including, e.g., a ventilator, a surgical tool, a catheter, or an intravenous catheter. In some embodiments, a pharmaceutical composition comprising one or more of the anti-S. aureus alpha toxin antibodies or antigen binding fragments thereof described herein is useful in subjects that are mechanically ventilated.

In some embodiments, a pharmaceutical composition comprising one or more of the anti-S. aureus alpha toxin antibodies or antigen binding fragments thereof described herein is useful in subjects colonized with S. aureus, e.g., in the lower respiratory tract.

In some embodiments, the pharmaceutical composition comprises an amount of anti-S. aureus alpha toxin antibodies or antigen binding fragments thereof that is effective to inhibit growth of bacteria in a subject. In some embodiments, the bacteria is S. aureus.

In some embodiments, the methods of treating, preventing and/or ameliorating a condition associated with a bacterial infection, e.g., an S. aureus infection, comprises contacting a subject infected with S. aureus with a pharmaceutical composition comprising anti-S. aureus alpha toxin antibodies or antigen binding fragments thereof in vivo. In some embodiments, a pharmaceutical composition comprising an anti-S. aureus alpha toxin antibody or antigen binding fragment thereof is administered at the same time or shortly after a subject has been exposed to bacteria to prevent infection. In some embodiments, the pharmaceutical composition comprising an anti-S. aureus alpha toxin antibody or antigen binding fragment thereof is administered as a therapeutic after infection.

In certain embodiments, the method of treating, preventing, and/or ameliorating bacterial infections, e.g., S. aureus infections, comprises administering to a subject a pharmaceutical composition comprising an anti-S. aureus alpha toxin antibody or antigen binding fragment thereof. In certain embodiments, the subject is a human. In some embodiments, the pharmaceutical composition comprising an anti-S. aureus alpha toxin antibody or antigen binding fragment thereof is administered before the subject is infected with S. aureus. In some embodiments, the pharmaceutical composition comprising an anti-S. aureus alpha toxin antibody or antigen binding fragment thereof is administered after the subject is infected with S. aureus.

In certain embodiments, the pharmaceutical composition comprising an anti-S. aureus alpha toxin antibody or antigen binding fragment thereof is administered to a subject on a ventilator. In certain embodiments, the subject has a catheter (e.g., a urinary catheter or an intravenous catheter). In certain embodiments, the subject is receiving antibiotics.

In certain embodiments, a pharmaceutical composition comprising an anti-S. aureus alpha toxin antibody or antigen binding fragment thereof is for the treatment or prevention of a nosocomial bacterial infection, e.g., a nosocomial S. aureus infection. In certain embodiments, a pharmaceutical composition comprising an anti-S. aureus alpha toxin antibody or antigen binding fragment thereof is for the treatment or prevention of an opportunistic bacterial infection, e.g., an opportunistic S. aureus infection. In certain embodiments, a pharmaceutical composition comprising an anti-S. aureus alpha toxin antibody or antigen binding fragment thereof is for the treatment or prevention of a bacterial infection, e.g., an S. aureus infection, following an organ transplant.

In certain embodiments, a pharmaceutical composition comprising anti-S. aureus alpha toxin antibodies or antigen binding fragments thereof is for the treatment or prevention of a methicillin resistant bacterial (e.g., S. aureus) infection. In certain embodiments, a pharmaceutical composition comprising anti-S. aureus alpha toxin antibodies or antigen binding fragments thereof is for the treatment or prevention of a glycopeptide intermediately resistant bacterial (e.g., S. aureus) infection. In certain embodiments, a pharmaceutical composition comprising anti-S. aureus alpha toxin antibodies or antigen binding fragments thereof is for the treatment or prevention of a glycopeptide resistant bacterial (e.g., S. aureus) infection.

For the treatment, prevention and/or amelioration of a condition associated with a bacterial infection, e.g., an S. aureus infection, the pharmaceutical composition or anti-S. aureus alpha toxin antibodies or antigen binding fragments thereof can be administered one time or over a series of treatments lasting from several days to several months, or until a cure is effected or a diminution of the condition is achieved. Optimal dosing schedules can be calculated from measurements of drug accumulation in the body of the patient and will vary depending on the relative potency of an individual antibody or agent. In some embodiments, the pharmaceutical composition or the anti-S. aureus alpha toxin antibody or antigen binding fragment thereof is administered one time.

In certain embodiments, a pharmaceutical composition comprises 1750 mg of anti-S. aureus alpha toxin antibodies or antigen binding fragments thereof. In certain embodiments, a pharmaceutical composition comprises 2000 mg of anti-S. aureus alpha toxin antibodies or antigen binding fragments thereof. In certain embodiments, a pharmaceutical composition comprises 2250 mg of anti-S. aureus alpha toxin antibodies or antigen binding fragments thereof. In certain embodiments, a pharmaceutical composition comprises 2500 mg of anti-S. aureus alpha toxin antibodies or antigen binding fragments thereof. In certain embodiments, a pharmaceutical composition comprises 2750 mg of anti-S. aureus alpha toxin antibodies or antigen binding fragments thereof. In certain embodiments, a pharmaceutical composition comprises 3000 mg of anti-S. aureus alpha toxin antibodies or antigen binding fragments thereof. In certain embodiments, a pharmaceutical composition comprises 3250 mg of anti-S. aureus alpha toxin antibodies or antigen binding fragments thereof. In certain embodiments, a pharmaceutical composition comprises 3500 mg of anti-S. aureus alpha toxin antibodies or antigen binding fragments thereof. In certain embodiments, a pharmaceutical composition comprises 3750 mg of anti-S. aureus alpha toxin antibodies or antigen binding fragments thereof. In certain embodiments, a pharmaceutical composition comprises 4000 mg of anti-S. aureus alpha toxin antibodies or antigen binding fragments thereof. In certain embodiments, a pharmaceutical composition comprises 4250 mg of anti-S. aureus alpha toxin antibodies or antigen binding fragments thereof. In certain embodiments, a pharmaceutical composition comprises 4500 mg of anti-S. aureus alpha toxin antibodies or antigen binding fragments thereof. In certain embodiments, a pharmaceutical composition comprises 4750 mg of anti-S. aureus alpha toxin antibodies or antigen binding fragments thereof. In certain embodiments, a pharmaceutical composition comprises 5000 mg of anti-S. aureus alpha toxin antibodies or antigen binding fragments thereof.

### IV. Methods of Administration

The anti-S. aureus alpha toxin antibodies or antigen binding fragments thereof described herein are useful in a variety of applications including, but not limited to, pneumonia, bacteremia, bone and joint infections, deep skin or tissue infection, meningitis, endocarditis due to bacterial infection (e.g., S. aureus infection). In some embodiments, the anti-S. aureus alpha toxin antibodies or antigen binding fragments thereof described herein are useful in nosocomial infections, opportunistic infections, infections following organ transplants, and other conditions associated with a bacterial infection (e.g. infection with S. aureus). In some embodiments, the anti-S. aureus alpha toxin antibodies or antigen binding fragments thereof are useful in subjects exposed to a bacterially contaminated device, including, e.g., a ventilator, a surgical tool, a catheter, or an intravenous catheter. In some embodiments, the anti-S. aureus alpha toxin antibodies or antigen binding fragments thereof are useful in mechanically ventilated subjects.

In some embodiments, the disclosure provides methods of treating, preventing and/or ameliorating a condition associated with a bacterial infection (e.g., an S. aureus infection) comprising administering an effective amount of an anti-S. aureus alpha toxin antibody or antigen binding fragment thereof to a subject. In some embodiments, the amount is effective to inhibit growth of the bacteria (e.g., S. aureus) in the subject. In some embodiments, the subject has been exposed to S. aureus. In some embodiments, S. aureus has been detected in the subject. In some embodiments, the subject is suspected of being infected with S. aureus, e.g., based on symptoms.

In some embodiments, the disclosure further provides methods of inhibiting growth of bacteria comprising administering an anti-S. aureus alpha toxin antibody or antigen binding fragment thereof to a subject. In some embodiments, the bacteria is S. aureus. In some embodiments, the subject has been exposed to S. aureus. In some embodiments, S. aureus has been detected in the subject. In some embodiments, the subject is suspected of being infected with a S. aureus, e.g., based on symptoms.

In certain embodiments, the subject is a human.

In some embodiments, the effective amount of anti-S. aureus alpha toxin antibodies or antigen binding fragments thereof is administered before the subject is infected with a bacteria (e.g., S. aureus). In some embodiments, the effective amount of anti-S. aureus alpha toxin antibodies or antigen binding fragments thereof is administered after the subject is infected with a bacteria (e.g., S. aureus).

In certain embodiments, the subject is on a ventilator. In certain embodiments, the subject has a catheter (e.g., a urinary catheter or an intravenous catheter). In certain embodiments, the subject is receiving antibiotics. In certain embodiments, the subject is receiving vancomycin.

In certain embodiments, the bacterial infection (e.g., S. aureus infection) is a nosocomial infection. In certain embodiments, the infection (e.g., an S. aureus infection) is an opportunistic infection. In certain embodiments, the infection (e.g., an S. aureus infection) follows an organ transplant.

In certain embodiments, the bacteria (e.g., S. aureus) is methicillin resistant. In certain embodiments, the bacteria (e.g., S. aureus) is glycopeptide intermediately resistant. In certain embodiments, the bacteria (e.g., S. aureus) is glycopeptide resistant.

In certain embodiments, the method of treating, preventing, and/or ameliorating S. aureus infections comprises administering to a subject an effective amount of an anti-S. aureus alpha toxin antibody or antigen binding fragment thereof and an antibiotic. The anti-S. aureus alpha toxin antibodies or antigen binding fragments thereof and the antibiotic can be administered simultaneously or sequentially. The anti-S. aureus alpha toxin antibodies or antigen binding fragments thereof and the antibiotic can be administered in the same pharmaceutical composition. The anti-S. aureus alpha toxin antibodies or antigen binding fragments thereof and the antibiotic can be administered in separate pharmaceutical compositions simultaneously or sequentially. The antibiotic can be, for example, vancomycin.

According to the methods described herein, the anti-S. aureus alpha toxin antibodies or antigen binding fragments thereof can be administered at particular dosages. Anti-S. aureus alpha toxin antibodies or antigen binding fragments thereof provided herein are safe and effective in treating or preventing infections, in decreasing the severity of infections, and in inhibiting bacterial growth at doses of about 2000 to about 5000 mg. Thus, in some embodiments, the anti-S. aureus alpha toxin antibodies or antigen binding fragments thereof are administered at a dose of about 2000 mg. In some embodiments, the anti-S. aureus alpha toxin antibodies or antigen binding fragments thereof are administered at a dose of about 2250 mg. In some embodiments, the anti-S. aureus alpha toxin antibodies or antigen binding fragments thereof are administered at a dose of about 2500 mg. In some embodiments, the anti-S. aureus alpha toxin antibodies or antigen binding fragments thereof are administered at a dose of about 2750 mg. In some embodiments, the anti-S. aureus alpha toxin antibodies or antigen binding fragments thereof are administered at a dose of about 3000 mg. In some embodiments, the anti-S. aureus alpha toxin antibodies or antigen binding fragments thereof are administered at a dose of about 3250 mg. In some embodiments, the anti-S. aureus alpha toxin antibodies or antigen binding fragments thereof are administered at a dose of about 3500 mg. In some embodiments, the anti-S. aureus alpha toxin antibodies or antigen binding fragments thereof are administered at a dose of about 3750 mg. In some embodiments, the anti-S. aureus alpha toxin antibodies or antigen binding fragments thereof are administered at a dose of about 4000 mg. In some embodiments, the anti-S. aureus alpha toxin antibodies or antigen binding fragments thereof are administered at a dose of about 4250 mg. In some embodiments, the anti-S. aureus alpha toxin antibodies or antigen binding fragments thereof are administered at a dose of about 4500 mg. In some embodiments, the anti-S. aureus alpha toxin antibodies or antigen binding fragments thereof are administered at a dose of about 4750 mg. In some embodiments, the anti-S. aureus alpha toxin antibodies or antigen binding fragments thereof are administered at a dose of about 5000 mg.

In some embodiments, the anti-S. aureus alpha toxin antibodies or antigen binding fragments thereof are administered at a dose of about 2000 to about 5000 mg. In some embodiments, the anti-S. aureus alpha toxin antibodies or antigen binding fragments thereof are administered at a dose of about 2000 to about 4500 mg. In some embodiments, the anti-S. aureus alpha toxin antibodies or antigen binding fragments thereof are administered at a dose of about 2000 to about 4000 mg. In some embodiments, the anti-S. aureus alpha toxin antibodies or antigen binding fragments thereof are administered at a dose of about 2000 to about 3000 mg.

In some embodiments, the anti-S. aureus alpha toxin antibodies or antigen binding fragments thereof are administered at a dose of about 3000 to about 5000 mg. In some embodiments, the anti-S. aureus alpha toxin antibodies or antigen binding fragments thereof are administered at a dose of about 4000 to about 5000 mg.

The anti-S. aureus alpha toxin antibodies or antigen binding fragments can be administered in any number of ways including systemic treatment. In some embodiments, the anti-S. aureus alpha toxin antibodies or antigen binding fragments are administered intravenously.

In some embodiments, the serum target level of anti-S. aureus alpha toxin antibodies or antigen binding fragments thereof is in a range of about 100 μg/ml to about 1000 μg/ml, about 200 μg/ml to about 500 μg/ml, about 250 μg/ml to about 450 μg/ml, about 200 μg/ml to about 300 μg/ml, about 211 μg/ml to about 1000 μg/ml, about 211 μg/ml to about 500 μg/ml, about 211 μg/ml to about 400 μg/ml, about 211 μg/ml to about 300 μg/ml, or at about 200 μg/ml, at about 210 μg/ml, at about 220 μg/ml, at about 230 μg/ml, at about 240 μg/ml, at about 250 μg/ml, or at about 300 μg/ml. In one embodiment, the serum target level of anti-S. aureus alpha toxin antibodies or antigen binding fragments thereof is at least 211 μg/ml.

In some embodiments, administration of the anti-S. aureus alpha toxin antibodies or antigen binding fragments thereof produces a serum target level of at least 211 μg/ml.

In some embodiments, administration of the anti-S. aureus alpha toxin antibodies or antigen binding fragments thereof prevents or treats a nosocomial infection (e.g., an S. aureus) infection. In some embodiments, administration of the anti-S. aureus alpha toxin antibodies or antigen binding fragments thereof decreases the severity of a nosocomial infection (e.g., an S. aureus) infection.

In some embodiments, administration of the anti-S. aureus alpha toxin antibodies or antigen binding fragments thereof prevents or treats pneumonia (e.g., pneumonia caused by S. aureus). In some embodiments, administration of the anti-S. aureus alpha toxin antibodies or antigen binding fragments decreases the severity of pneumonia (e.g., pneumonia caused by S. aureus). In some embodiment, the pneumonia is ventilator associated pneumonia. In some embodiments, the pneumonia is pneumonia following extubation.

In some embodiments, administration of the anti-S. aureus alpha toxin antibodies or antigen binding fragments thereof reduces pneumonia (e.g., pneumonia caused by S. aureus) as compared to the current standard of care which can include one or more of the following approaches: elevation of the head of the bed, daily “sedation vacations,” peptic ulcer disease propylaxis, deep venous thrombosis prophylaxis and daily oral care with cholohexidine.

In some embodiments, administration of the anti-S. aureus alpha toxin antibodies or antigen binding fragments thereof reduces ventilator associated pneumonia (e.g., caused by S. aureus) as compared to the current standard of care which can also include one or more of the following approaches: elevation of the head of the bed, daily “sedation vacations,” peptic ulcer disease propylaxis, deep venous thrombosis prophylaxis and daily oral care with cholohexidine.

In some embodiments, administration of the anti-S. aureus alpha toxin antibodies or antigen binding fragments thereof decreases nasal and/or tracheal bacterial (e.g. S. aureus) colonization.

In some embodiments, administration of the anti-S. aureus alpha toxin antibodies or antigen binding fragments thereof reduces morbidity. Morbidity can be measured by health care resource utilization (e.g., days on ventilator, length of ICU stay, length of hospital stay, and/or increased antibiotic-free days). In some embodiments, administration of the anti-S. aureus alpha toxin antibodies or antigen binding fragments thereof to a patient with pneumonia increase antibiotic-free days. In some embodiments, administration of the anti-S. aureus alpha toxin antibodies or antigen binding fragments thereof to a patient with pneumonia reduces days on ventilator. In some embodiments, administration of the anti-S. aureus alpha toxin antibodies or antigen binding fragments thereof to a patient with pneumonia reduces length of ICU stay. In some embodiments, administration of the anti-S. aureus alpha toxin antibodies or antigen binding fragments thereof to a patient with pneumonia reduces length of hospital stay.

In some embodiments, administration of the anti-S. aureus alpha toxin antibodies or antigen binding fragments thereof reduces mortality.

In some embodiments, administration of the anti-S. aureus alpha toxin antibodies or antigen binding fragments thereof prevents or treats bacteremia (e.g., caused by S. aureus). In some embodiments, administration of the anti-S. aureus alpha toxin antibodies or antigen binding fragments thereof prevents or treats bone infection (e.g., caused by S. aureus). In some embodiments, administration of the anti-S. aureus alpha toxin antibodies or antigen binding fragments thereof prevents or treats joint infection (e.g., caused by S. aureus). In some embodiments, administration of the anti-S. aureus alpha toxin antibodies or antigen binding fragments thereof prevents or treats bone and joint infection (e.g., caused by S. aureus). In some embodiments, administration of the anti-S. aureus alpha toxin antibodies or antigen binding fragments thereof prevents or treats sepsis (e.g., caused by S. aureus). In some embodiments, administration of the anti-S. aureus alpha toxin antibodies or antigen binding fragments thereof prevents or treats deep tissue infection (e.g., caused by S. aureus). In some embodiments, administration of the anti-S. aureus alpha toxin antibodies or antigen binding fragments thereof prevents or treats meningitis (e.g., caused by S. aureus).

In some embodiments, administration of the anti-S. aureus alpha toxin antibodies or antigen binding fragments thereof decreases the severity of bacteremia (e.g., caused by S. aureus). In some embodiments, administration of the anti-S. aureus alpha toxin antibodies or antigen binding fragments thereof decreases the severity of bone infection (e.g., caused by S. aureus). In some embodiments, administration of the anti-S. aureus alpha toxin antibodies or antigen binding fragments thereof decreases the severity of joint infection (e.g., caused by S. aureus). In some embodiments, administration of the anti-S. aureus alpha toxin antibodies or antigen binding fragments thereof decreases the severity of bone and joint infection (e.g., caused by S. aureus). In some embodiments, administration of the anti-S. aureus alpha toxin antibodies or antigen binding fragments thereof decreases the severity of sepsis (e.g., caused by S. aureus). In some embodiments, administration of the anti-S. aureus alpha toxin antibodies or antigen binding fragments thereof decreases the severity of tissue infection (e.g., caused by S. aureus). In some embodiments, administration of the anti-S. aureus alpha toxin antibodies or antigen binding fragments thereof decreases the severity of meningitis (e.g., caused by S. aureus).

### Example 1: Pharmacokinetics (PK) and Pharmacodynamics (PD) of MEDI4893

Pharmacokinetics (PK) and pharmacodynamics (PD) of MEDI4893 in a Staphylococcus aureus-induced mouse pneumonia model following a prophylactic administration was evaluated. Alpha toxin (AT) is a major virulence factor for respiratory infections caused by S. aureus (Bubeck-Wardenburg et. al., 2007; Bubeck-Wardenburg et. al., 2008). Anti-AT mAb MEDI4893 and LC10 reduce disease severity through neutralization of AT activity. MEDI4893 and LC10 share the same protein sequence except for the YTE mutations in the Fc region. The YTE mutation extends antibody half-life in humans without affecting binding affinity to target. In contrast to the extension of half life in humans, YTE mutation shortens antibody half life in mice. For this reason, LC10 was used to evaluate PK/PD property of MEDI4893 in mice.

The study was conducted in normal healthy C57BL6/J mice and in C57 BL6/J mice intra-nasally infected with S. aureus one day post intraperitoneal (IP) dosing of LC10. Anti-S. aureus infection effect of LC10 was also compared to that of a control human antibody R347 that does not bind to AT. A wide dose range (3-14.3 mg/kg) of LC10 was studied in control and infected mice. Serum, lung and BAL samples were collected at various time-points up to 28 days post-dose for determining total and free LC10 concentrations and total and free AT concentrations in serum, lung and BAL.

Following a single IP injection, total LC10 concentrations in serum increased as dose increased. Peak concentration, Cₘₐₓ, increased from 32.7 μg/mL in the 3 mg/kg dose group to 211 μg/mL in the 14.3 mg/kg dose group. The increase in area under the curve over the observation period (AUCₗₐₛₜ) in non-infected control mice appeared approximately dose-proportional (451 μg·day/mL at 3 mg/kg and 2460 μg·day/mL at 14.3 mg/kg), indicating linear PK. Total LC10 exposure in infected mice was comparable to control mice during the first 5 days but slightly lower than control mice thereafter. In infected mice, free LC10 concentrations in serum appeared similar to total LC10 levels.

Total LC10 concentrations were much lower in the lungs and BAL than in serum (1-10% in the lungs and 0.1-10% in BAL) but increased as LC10 dose increased. At the highest dose (14.3 mg/kg), total LC10 in the lungs in infected mice appeared comparable to control mice. At the low doses (3 and 4.3 mg/kg), however, total LC10 levels were lower in infected mice than in control mice. Total LC10 in BAL was higher in infected mice than in control mice. Free LC10 concentrations in the lungs and BAL were in general similar to total LC10 in the lungs and BAL.

Total AT concentrations were much higher in the lungs than in serum. At 4.3 mg/kg group, for example, peak AT concentrations in the lungs and serum were 1791 ng/g and 277 ng/mL, respectively. Faster suppression of free AT in the lungs was seen as LC10 dose increased. Free AT in the lungs dropped below LLOQ values within 3 days at 14.3 mg/kg where it was still detectable 14 days post-dose at 3 and 4.3 mg/kg. AT in BAL decreased as LC10 dose increased. Peak free AT in BAL decreased from 2635 ng/mL at 3 mg/kg to 275 ng/mL at 14.3 mg/kg, indicating a dose dependent suppression of AT at the infection site.

In summary, LC10 exhibited linear PK. LC10 exposure in the lungs and BAL increased as dose increased. AT levels at infection site BAL decreased as LC10 dose increased. The above PK/PD results were utilized to design the study for clinical assessment of the safety and efficacy of MEDI4893, described in Example 2.

### Bioanalytical Methods

(A) Determination of Total LC10 Serum Concentration

The concentration of total LC10 in mouse serum samples was determined using a qualified enzyme-linked immunosorbent assay (ELISA) method as described below.

The method procedure is a heterogenous format in which wash steps follow each incubation. A microtiter plate is first coated with 0.5 μg/mL sheep anti-human IgG (H+L) followed by a block step. Calibrators, quality controls, and samples, diluted appropriately in the assay buffer (Phosphate Buffered Saline+0.5% BSA+0.1% Tween-20), are added to the blocked microtiter plate and incubated for 1.5 hours. The secondary detection reagent, goat anti-human IgG (H+L)-HRP, is added after 1:15,000 dilution and incubated for 30 minutes. TMB (3,3′,5,5′-tetramethylbenzidine) substrate is used to quantitatively measure the binding complex; chromogenic color development is directly related to levels of analyte in the sample. Once stopped with acid, the plate is read on a spectrophotometer at 450 nm, and data analyzed with SoftMax® Pro (SMP), version 5.4 or higher. The standard curve was established using a four parameter logistical curve fit model without weighting provided by the software program SoftMax Pro v5.4. The nominal range of this assay is 1-100 ng/mL.

(B) Determination of Free LC10 Serum Concentration

The concentration of free LC10 in mouse skin samples was determined using a qualified enzyme-linked immunosorbent assay (ELISA) method as described.

The method procedure is a heterogenous format in which wash steps follow each incubation. A microtiter plate is first coated with 1.5 μg/mL native alpha toxin (AT) followed by a block step. Calibrators, quality controls, and samples, diluted appropriately in the assay buffer (Phosphate Buffered Saline+0.5% BSA+0.1% Tween-20), are added to the blocked microtiter plate and incubated for 1.5 hours. The primary detection reagent, biotin-labeled AT, is added and incubated for 1 hour. The secondary detection reagent, streptavidin-HRP, is added after 1:15,000 dilution and incubated for 30 minutes. TMB (3,3′,5,5′-tetramethylbenzidine) substrate is used to quantitatively measure the binding complex; chromogenic color development is directly related to levels of analyte in the sample. Once stopped with acid, the plate is read on a spectrophotometer at 450 nm, and data analyzed with SoftMax® Pro (SMP), version 5.4 or higher. The standard curve was established using a four parameter logistical curve fit model without weighting provided by the software program SoftMax Pro v5.4. The nominal range of this assay is 0.3-15 ng/mL.

(C) Determination of Total LC10 Lung Concentration

The concentration of total LC10 in mouse lung homogenates samples was determined using a qualified enzyme-linked immunosorbent assay (ELISA) method as described.

The method procedure is a heterogeneous format in which wash steps follow each incubation. A microtiter plate is first coated with 0.5 pig/mL sheep anti-human IgG (H+L) followed by a block step. Calibrators, quality controls, and samples, diluted appropriately in the assay buffer (Phosphate Buffered Saline+0.5% BSA+0.1% Tween-20), are added to the blocked microtiter plate and incubated for 1.5 hours. The secondary detection reagent, goat anti-human IgG (H+L)-HRP, is added after 1:15,000 dilution and incubated for 30 minutes. TMB (3,3′,5,5′-tetramethylbenzidine) substrate is used to quantitatively measure the binding complex; chromogenic color development is directly related to levels of analyte in the sample. Once stopped with acid, the plate is read on a spectrophotometer at 450 nm, and data analyzed with SoftMax® Pro (SMP), version 5.4 or higher. The standard curve was established using a four parameter logistical curve fit model without weighting provided by the software program SoftMax Pro v5.4. The nominal range of this assay is 0.1-10 μg/mL.

(D) Determination of Free LC10 Lung Concentration

The concentration of free LC10 in mouse Lung Homogenate Lysate samples was determined using a qualified enzyme-linked immunosorbent assay (ELISA) method as described below.

The method procedure is a heterogeneous format in which wash steps follow each incubation. A microtiter plate is first coated with 1.5 μg/mL native alpha toxin (AT), followed by a block step. Calibrators, quality controls, and samples, diluted appropriately in the assay buffer (Phosphate Buffered Saline+0.5% BSA+0.1% Tween-20), are added to the blocked microtiter plate and incubated for 1.5 hours. Primary detection antibody, biotin-labeled AT is added at 1:760 dilution and incubated for 1 hour, then secondary detection reagent, streptavidin-HRP is added after 1:15,000 dilution and incubated for 30 minutes. TMB (3,3′,5,5′-tetramethylbenzidine) substrate is used to quantitatively measure the binding complex; chromogenic color development is directly related to levels of analyte in the sample. Once stopped with acid, the plate is read on a spectrophotometer at 450 nm, and data analyzed with SoftMax® Pro (SMP), version 5.4 or higher. The standard curve was established using a four parameter logistical curve fit model without weighting provided by the software program SoftMax Pro v5.4. The nominal range of this assay is 0.03-1.5 μg/mL.

(E) Determination of Total LC10 BAL Concentration

The concentration of total LC10 in mouse BAL Lysate samples was determined using a qualified enzyme-linked immunosorbent assay (ELISA) method as described.

The method procedure is a heterogeneous format in which wash steps follow each incubation. A microtiter plate is first coated with 0.5 μg/mL sheep anti-human IgG (H+L) followed by a block step. Calibrators, quality controls, and samples, diluted appropriately in the assay buffer (Phosphate Buffered Saline+0.5% BSA+0.1% Tween-20), are added to the blocked microtiter plate and incubated for 1.5 hours. The secondary detection reagent, goat anti-human IgG (H+L)-HRP, is added after 1:15,000 dilution and incubated for 30 minutes. TMB (3,3′,5,5′-tetramethylbenzidine) substrate is used to quantitatively measure the binding complex; chromogenic color development is directly related to levels of analyte in the sample. Once stopped with acid, the plate is read on a spectrophotometer at 450 nm, and data analyzed with SoftMax® Pro (SMP), version 5.4 or higher. The standard curve was established using a four parameter logistical curve fit model without weighting provided by the software program SoftMax Pro v5.4. The nominal range of this assay is 0.1-10 μg/mL.

(F) Determination of Free LC10 BAL Concentration

The concentration of free LC10 in mouse BAL lysate samples was determined using a qualified enzyme-linked immunosorbent assay (ELISA) method as described.

The method procedure is a heterogeneous format in which wash steps follow each incubation. A microtiter plate is first coated with 1.5 μg/mL native alpha toxin (AT), followed by a block step. Calibrators, quality controls, and samples, diluted appropriately in the assay buffer (Phosphate Buffered Saline+0.5% BSA+0.1% Tween-20), are added to the blocked microtiter plate and incubated for 1.5 hours. Primary detection antibody, biotin-labeled AT is added at 1:760 dilution and incubated for 1 hour, then secondary detection reagent, streptavidin-HRP is added after 1:15,000 dilution and incubated for 30 minutes. TMB (3,3′,5,5′-tetramethylbenzidine) substrate is used to quantitatively measure the binding complex, chromogenic color development is directly related to levels of analyte in the sample. Once stopped with acid, the plate is read on a spectrophotometer at 450 nm, and data analyzed with SoftMax® Pro (SMP), version 5.4 or higher. The standard curve was established using a four parameter logistical curve fit model without weighting provided by the software program SoftMax Pro v5.4. The nominal range of this assay is 0.03-1.5 μg/mL.

(G) Determination of Total Alpha Toxin Concentrations

A 96 well microtiter plate is first coated with rabbit anti-AT IgG diluted to 1 μg/mL in PBS and 50 μL (100 μL for lung) were added in to each well. The plates were incubated over night at 4° C. After incubation plates were washed 4-times by adding 200 μL wash buffer (PBS+0.1% tween 20) to each well. Twenty-five microliter (50 μL for lung) of standard controls (0.781 ng/mL-100 ng/mL) and test samples diluted appropriately in the assay buffer (PBS pH7.4) were added to the microtiter plate. The plates were incubated at room temperature for 1 hr±10 minutes. Following incubation, the plates were washed 4 times and incubated at room temperature for 1 hr±10 minutes with 25 μL (50 μL for lung) LC10 (100 ng/ml). Following washing, 25 μL (50 μL for lung) goat anti-human Fcγ-HRP was added at 1:8,000 dilution. The plates were incubated at room temperature for 1 hr±10 minutes and washed again as described above. TMB (3,3′,5,5′-tetramethylbenzidine) substrate 25 μL/well (50 μL/well for lung) was added in to the plates and incubated room temperature in the dark for 15 minutes. The reaction was stopped by the addition of 25 μL (50 μL for lung) of 0.2M H2SO4 to each well. Once stopped with acid, the plate is read on a spectrophotometer at 450 nm, and data analyzed with Soft Max® Pro (SMP), version 5.4 or higher. The standard curve was established using a four parameter logistical curve fit model without weighting provided by the software program Soft Max Pro v5.4. The lower limit of quantitation (LLOQ) was >1 ng/mL.

(H) Determination of Free Alpha Toxin Concentrations

A 96 well microtiter plate is first coated with rabbit anti-AT IgG diluted to 1 μg/mL in PBS and 50 μl (100 μL for lung) were added in to each well. The plates were incubated over night at 4° C. After incubation plates were washed 4-times by adding 200 μL wash buffer (PBS+0.1% tween 20) to each well. Twenty-five microliter (50 μL for lung) of standard controls (0.781 ng/mL-100 ng/mL) and test samples diluted appropriately in the assay buffer (PBS pH7.4) were added to the microtiter plate. The plates were incubated at room temperature for 1 hr±10 minutes. Following incubation, the plates were washed 4 times and incubated at room temperature for 1 hr±10 minutes with 25 μL (50 μL for lung) HRP conjugated LC10 (100 ng/mL). Following washing, TMB (3,3′,5,5′-tetramethylbenzidine) substrate 25 μl/well (50 μL/well for lung) was added in to the plates and incubated at room temperature in the dark for 15 minutes. The reaction was stopped by the addition of 25 μL (50 μL for lung) of 0.2M H2SO4 to each well. Wait approximately 5 minutes before reading the plate. Once stopped with acid, the plate is read on a spectrophotometer at 450 nm, and data analyzed with Soft Max® Pro (SMP), version 5.4 or higher. The standard curve was established using a four parameter logistical curve fit model without weighting provided by the software program Soft Max Pro v5.4. The lower limit of quantitation (LLOQ) was >1 ng/mL.

### Pharmacokinetic Evaluations and Pharmacodynamic Analysis

Non-compartmental pharmacokinetic analysis was performed using WinNonlin Professional (version 5.2, Pharsight Corp., Mountain View, Calif.). Nominal collection times were used for the PK data analyses. The BLQ values were set to missing for the NCA analysis.

Cmax and Tmax were observed values. AUC from time zero to time of the last quantifiable sample, AUClast, was calculated using the linear/log trapezoidal method as implemented in WinNonlin Professional (version 5.2, Pharsight, Corp., Mountain View, Calif.). Summary statistics for free and total alpha toxin levels in serum and skin were calculated using WinNonlin Professional (version 5.2, Pharsight Corp., Mountain View, Calif.).

### Example 2: Clinical Assessment of the Safety and Efficacy of MEDI4893

(A) Subjects

Approximately 462 subjects are enrolled in centers primarily in Europe and randomized into one of three treatment groups: 2000 mg MEDI4893 (N=154), 5000 mg MEDI4893 (N=154), or placebo (N=154). Randomization is stratified based on country and then by whether or not subjects received anti-S. aureus systemic antibiotic (treatment for no more than 24 hours) within the 48 hours prior to randomization to ensure that no more than about 75% of the study population consists of subjects in either stratification level of prior anti-S. aureus systemic antibiotic treatment.

In order to be enrolled, subjects are required to be 18 years of age or older at the time of study entry and have a tracheal or bronchial sample positive for S. aureus within 36 hours prior to randomization. Subjects must also be currently intubated on mechanical ventilation in the intensive care unit (ICU) and expected to remain intubated and mechanically ventilated for at least three days. Subjects with evidence of resolved pneumonia can be enrolled, but subjects with diagnosis of new-onset pneumonia within 72 hours prior to randomization cannot be enrolled.

Subjects are not eligible to participate if they have acute confirmed or suspected Staphylococcal disease at enrollment and have received MEDI4893. Subjects are also not eligible to participate if they have a clinical pulmonary infection score (CPIS) of six or greater within the past 24 hours prior to MEDI4893 dosing. Subjects with active pulmonary disease that would impair the ability to diagnosis pneumonia (e.g., active tuberculosis or fungal disease, obstructing lung cancer, cystic fibrosis) are not eligible. Subjects are also not eligible to participate if they have been on mechanical ventilation for more than 2 weeks and are known to be colonized with S. aureus in the lower respiratory tract for more than 2 weeks. Subjects who are tracheostomy-dependent prior to hospital admission and subjects with burns on more than 40% of body surface area are not eligible. In addition, subjects are not eligible if they received an anti-S. aureus systemic antibiotic for more than 24 hours within 48 hours prior to randomization, wherein the antibiotic is considered active against the S. aureus strain with which the subject is colonized, or wherein ongoing receipt of the anti-S. aureus systemic antibiotic is anticipated.

(B) Design of the Study

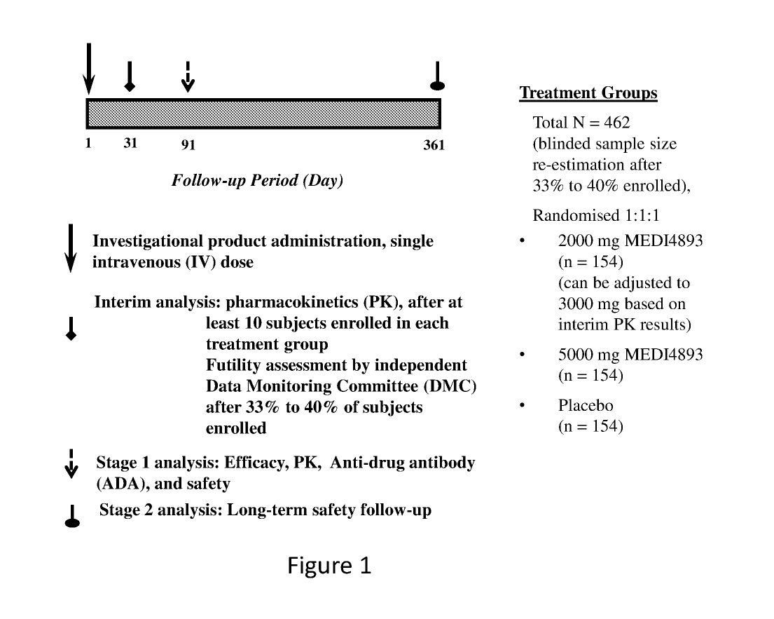

A phase 2 randomized, double-blind, placebo-controlled study is conducted on 462 subjects. A flow diagram of the study is provided in FIG. 1. Subjects are monitored for clinical symptoms of pneumonia and other serious S. aureus infection at screening.

At screening, this monitoring includes a physical examination, evaluation of vital signs, Clinical Pulmonary Infection Score (CPIS) assessment, Sequential Organ Failure Assessment (SOFA), Acute Physiological and Chronic Health Evaluation (APACHE)-IL, Glasgow Coma Scale (GCS), and PaO₂/FiO₂ ratio. For both the CPIS assessment and SOFA, the last available chest X-ray, serum chemistry and/or complete blood count (CBC) values obtained within 72 hours can be used in determining the score.

In addition, tracheal or bronchial aspirate samples to assess S. aureus colonization are collected.

Within 72 hours of being monitored for clinical symptoms and within 36 hours of the collection of the tracheal or bronchial aspirate, subjects are randomized into one of the three treatment groups: to receive a single intravenous (IV) infusion of either 2000 mg MEDI4893, 5000 mg MEDI4893, or placebo on Day 1. The subjects are followed through Day 361.

Subjects receive MEDI4893 or placebo solution intravenously through a dedicated line (either central or peripheral) through a low-protein binding 0.22-μm inline filter using an IV infusion pump over a minimum duration of 270 minutes.

On day 1 at the time of dose, and post-dose, subjects are monitored for clinical symptoms of pneumonia and other serious S. aureus infection. The monitoring includes a physical examination and evaluation of vital signs.

Two interim analyses are performed. One interim analysis occurs after at least 10 subjects from each treatment group are followed through 30 days post-dose for possible dose adjustment (e.g., an adjustment, for example, from the 2000 mg dose to a 3000 mg dose, in view of outcomes and serum levels of MEDI4893, can be made). Another interim analysis is conducted for futility assessment when approximately 33-40% of the enrolled patients are followed through 30 days post-dose.

(C) Safety and Efficacy Assessments

Two formal analyses (Stage 1 and Stage 2) are performed. During the Stage 1 analysis, efficacy, pharmacokinetic (PK), anti-drug antibody (ADA), and safety are analyzed on all data collected after the last subject has completed follow-up through 90 days post-dose. The Stage 2 analysis is performed after all subjects have completed the study, and it analyses the exploratory endpoints measuring pharmacoeconomic evaluations after Day 91 and safety through 360 days post-dose. Both the Stage 1 and Stage 2 analyses are performed to demonstrate that a single IV dose of MEDI4893 from 2000 to 5000 mg has an acceptable safety profile and is capable of reducing the incidence of S. aureus pneumonia in mechanically ventilated subjects in the Intensive Care Unit (ICU) who are colonized with S. aureus in the lower respiratory tract through 30 days post dose (irrespective of mechanical ventilation status at the time of diagnosis).

Blood and tracheal aspirate samples are collected for microbiological assessment of S. aureus colonization on Days 2, 8(±1 day), 15(±1 day), 22(±1 day), 31(±1 day), 61(±1 day), and 91 (±1 day), provided the subject remains intubated.

Subjects are monitored for clinical symptoms of pneumonia and other serious S. aureus infection daily while in hospital, and in accordance with symptoms after hospital discharge. At these times, the monitoring includes a physical examination and evaluation of vital signs. While in the hospital, CPIS is assessed daily while the subject remains on mechanical ventilation.

For subjects with suspected serious S. aureus infection, clinical symptoms (CPIS, SOFA, physical exam, and vital signs) are assessed at the day of onset and then daily through resolution. Blood samples are taken from all subjects with suspected serious S. aureus infection on the day of onset and the two following days. Blood sampling is repeated every other day in those that are positive for S. aureus pneumonia until they are negative for S. aureus. Tracheal or bronchial aspirates are taken from subjects with suspected serious S. aureus infection that are intubated on the day of onset and the two following days. Tracheal or bronchial aspirates are repeated every other day in those that are positive for S. aureus pneumonia until resolution. Expectorated sputum is analyzed in subjects with suspected serious S. aureus infection that are not intubated (unless bronchoscopy performed for clinical management and BAL or PSB sample available) on the day of onset and the two following days. Expectorated sputum analyses is repeated every other day in those that are positive for S. aureus pneumonia until resolution. Chest X-rays are performed on subjects with suspected serious S. aureus infection on the day of onset. Chest X-rays are repeated in those with confirmed pneumonia as clinically indicated through resolution.

In subjects who are mechanically ventilated at the time of diagnosis of S. aureus pneumonia, the criteria for the diagnosis requires that the subject demonstrate the following radiographic, clinical, and microbiologic new onset symptoms/signs that are not due to any other non-infectious cases.
Radiographic Criteria: new or worsening infiltrate consistent with pneumonia on chest X-ray obtained within 24 hours of the event
Clinical Criteria: at least 2 minor or 1 major respiratory signs or symptoms of new onset

Minor
Systemic signs of infection (one or more of the following): Abnormal temperature (oral or tympanic temperature>38° C. or a core temperature≥38.3° C. or hypothermia, defined as a core body temperature of <35° C.), and/or abnormal WBC (WBC count>10,000 cells/mm³, WBC count<4500 cells/mm³, or >15% band neutrophils)
Production of purulent endotracheal secretions
Auscultatory findings consistent with pneumonia/pulmonary consolidation (eg, rales, rhonchi, bronchial breath sounds, dullness to percussion)

Major
Acute changes made in the ventilatory support system to enhance oxygenation, as determined by (a) PaO₂/FiO₂ ratio<240 mmHg, or (b) decrease in PaO₂/FiO₂ by ≥50 mmHg
Microbiologic Confirmation: at least 1 of the following (obtained within 24 hours of onset of the event)
Respiratory specimen is positive for S aureus by culture. Includes a specimen of respiratory secretions obtained by endotracheal aspiration or by bronchoscopy with BAL or PSB sampling in intubated subjects
Blood culture positive for S aureus (and no apparent primary source of infection outside the lung)
Pleural fluid aspirate or lung tissue culture positive for S aureus during episode of pneumonia (only if obtained as part of the subject's necessary clinical management)

Subjects are considered mechanically ventilated if they are intubated with an endotracheal or nasotracheal tube and are receiving positive pressure ventilation support or if they are not intubated with an endotracheal or nasotracheal tube, but require 8 or more hours of positive pressure ventilation within the past 24 hours.

In subjects who are not mechanically ventilated at the time of diagnosis of S. aureus pneumonia, the criteria for the diagnosis requires that the subject demonstrate the following radiographic, clinical, and microbiologic new onset symptoms/signs that are not due to any other non-infectious cases.
Radiographic Criteria: new or worsening infiltrate consistent with pneumonia on chest X-ray obtained within 24 hours of the event
Clinical Criteria: at least 2 minor or 1 major respiratory signs or symptoms of new onset

Minor
Systemic signs of infection: Abnormal temperature (oral or tympanic temperature>38° C. or a core temperature≥38.3° C. or hypothermia, defined as a core body temperature of <35° C.), and/or abnormal WBC (WBC count>10,000 cells/mm³, WBC count<4500 cells/mm³, or >15% band neutrophils)
A new onset of cough (or worsening of cough)
Production of purulent sputum
Physical examination findings consistent with pneumonia/pulmonary consolidation such as auscultatory findings (e.g., rales, rhonchi, bronchial breath sounds), dullness to percussion, or pleuritic chest pain
Dyspnea, tachypnea (respiratory rate>30 breaths/minute), or hypoxemia defined as (a) O₂ saturation<90% or PaO₂<60 mmHg on room air if lower than baseline, or (b) A need to initiate or increase sustained (≥3 hours) supplemental oxygen to maintain pre-event baseline O₂ saturations

Major
A need to initiate non-invasive mechanical ventilation or re-initiate invasive mechanical ventilation because of respiratory failure or worsening of respiratory status
Microbiologic Confirmation: at least 1 of the following (obtained with 72 hours of onset of the event)
Respiratory specimen is positive for S aureus by culture. Includes either expectorated sputum or (only if obtained as part of the subject's necessary clinical management) a specimen of respiratory secretions obtained by bronchoscopy with BAL or PSB sampling. Respiratory samples from expectoration must show <10 squamous epithelial cells and >25 polymorphonuclear neutrophils per 100× field to be suitable
Blood culture positive for S aureus (and no other apparent primary source of infection outside the lung)
Pleural fluid aspirate or lung tissue culture positive for S aureus (only if obtained as part of the subject's necessary clinical management)

Subjects are considered not mechanically ventilated if they are not intubated with an endotracheal or nasotracheal tube or they require positive pressure ventilation support for less than 8 hours.

The incidence of S. aureus pneumonia through 30 days post dose is calculated to demonstrate that administration of 2000 to 5000 mg MEDI4893 reduces the incidence of S. aureus pneumonia. The incidence of S. aureus pneumonia through 30 days post dose will be compared in subjects on mechanical ventilation and subjects in whom mechanical ventilation is no longer required.

Adverse events and new onset chronic disease are reviewed to demonstrate that an administration of 2000 to 5000 mg MEDI4893 is safe.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of treating or preventing a nosocomial infection in a subject, or decreasing the severity of a nosocomial infection in a subject, comprising administering 2000 to 5000 milligrams of an anti-alpha toxin antibody or antigen-binding fragment thereof to the subject, wherein the antibody or antigen-binding fragment thereof comprises a set of Complementarity-Determining Regions (CDRs): HCDR1, HCDR2, HCDR3, LCDR1, LCDR2, and LCDR3 wherein,
HCDR1 has the amino acid sequence of SEQ. ID. NO: 1;
HCDR2 has the amino acid sequence of SEQ. ID. NO: 2;
HCDR3 has the amino acid sequence of SEQ. ID. NO: 3;
LCDR1 has the amino acid sequence of SEQ. ID. NO: 4;
LCDR2 has the amino acid sequence of SEQ. ID. NO: 5; and
LCDR3 has the amino acid sequence of SEQ. ID. NO: 6.

2. A method according to claim 1,
wherein the serum target level of said antibody or antigen-binding fragment thereof is in a range of about 100 μg/ml to about 1000 μg/ml.

3-9. (canceled)

10. A method of treating or preventing pneumonia in a subject comprising administering 2000 to 5000 milligrams of an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein,
HCDR1 has the amino acid sequence of SEQ. ID. NO: 1;
HCDR2 has the amino acid sequence of SEQ. ID. NO: 2;
HCDR3 has the amino acid sequence of SEQ. ID. NO: 3;
LCDR1 has the amino acid sequence of SEQ. ID. NO: 4;
LCDR2 has the amino acid sequence of SEQ. ID. NO: 5; and
LCDR3 has the amino acid sequence of SEQ. ID. NO: 6.

11. A method of treating or preventing pneumonia in a human subject comprising administering an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein,
HCDR1 has the amino acid sequence of SEQ. ID. NO: 1;
HCDR2 has the amino acid sequence of SEQ. ID. NO: 2;
HCDR3 has the amino acid sequence of SEQ. ID. NO: 3;
LCDR1 has the amino acid sequence of SEQ. ID. NO: 4;
LCDR2 has the amino acid sequence of SEQ. ID. NO: 5; and
LCDR3 has the amino acid sequence of SEQ. ID. NO: 6
wherein the serum target level of said antibody or antigen-binding fragment thereof is in a range of about 100 μg/ml to about 1000 μg/ml.

12. A method of decreasing the severity of pneumonia in a subject comprising administering 2000 to 5000 milligrams of an anti-alpha toxin antibody or antigen-binding fragment thereof to the subject, wherein the antibody or antigen-binding fragment thereof comprises a set of CDRs: HCDR1, HCDR2, HCDR3, LCDR1, LCDR2, and LCDR3 wherein,
HCDR1 has the amino acid sequence of SEQ. ID. NO: 1;
HCDR2 has the amino acid sequence of SEQ. ID. NO: 2;
HCDR3 has the amino acid sequence of SEQ. ID. NO: 3;
LCDR1 has the amino acid sequence of SEQ. ID. NO: 4;
LCDR2 has the amino acid sequence of SEQ. ID. NO: 5; and
LCDR3 has the amino acid sequence of SEQ. ID. NO: 6.

13-14. (canceled)

15. The method of claim 10, wherein the pneumonia is Staphylococcus aureus (S. aureus) pneumonia.

16. The method claim 10, wherein the pneumonia is ventilator associated pneumonia.

17. The method of claim 10, wherein the pneumonia is pneumonia following extubation.

18-19. (canceled)

20. The method of claim 15, wherein the S. aureus is resistant to methicillin.

21-22. (canceled)

23. The method of claim 10 wherein the subject is mechanically ventilated.

24-26. (canceled)

27. The method of claim 10, wherein the subject is colonized with S. aureus.

28-30. (canceled)

31. The method of claim 10, wherein the subject is free of S. aureus-related disease.

32. The method of claim 10, wherein the antibody or antigen-binding fragment thereof is administered intravenously.

33. The method of claim 10, wherein 2000 mg of the antibody or antigen-binding fragment thereof is administered.

34. (canceled)

35. The method of claim 10, wherein 3000 mg of the antibody or antigen-binding fragment thereof is administered.

36-38. (canceled)

39. The method of any one of claim 10, wherein 5000 mg of the antibody or antigen-binding fragment thereof is administered.

40. The method of claim 10, wherein the serum target level of the antibody or antigen-binding fragment thereof is in a range of about 200 μg/ml to about 500 μg/ml.

41. The method of claim 10, wherein the subject received an antibiotic prior to administration of the anti-alpha toxin antibody or antigen-binding fragment thereof.

42. The method of claim 10, wherein the anti-alpha toxin antibody or antigen-binding fragment thereof is co-administered with an antibiotic.

43-46. (canceled)

47. The method of claim 10, wherein the anti-alpha toxin antibody or antigen-binding fragment thereof comprises a heavy chain variable region (VH) comprising SEQ ID NO:7 and a light chain variable region (VL) comprising SEQ ID NO:8.

48. The method of claim 10, wherein the anti-alpha toxin antibody or antigen-binding fragment thereof comprises a YTE substitution in the constant region.

49-52. (canceled)


SEQUENCE LISTING (11 sequences)

SEQ ID NO: 1 - protein, 5 aa, Artificial Sequence
  Description of Artificial Sequence Synthetic peptide
SHDMH

SEQ ID NO: 2 - protein, 16 aa, Artificial Sequence
  Description of Artificial Sequence Synthetic peptide
GIGTAGDTYYPDSVKG

SEQ ID NO: 3 - protein, 14 aa, Artificial Sequence
  Description of Artificial Sequence Synthetic peptide
DRYSPTGHYYGMDV

SEQ ID NO: 4 - protein, 11 aa, Artificial Sequence
  Description of Artificial Sequence Synthetic peptide
RASQSISSWLA

SEQ ID NO: 5 - protein, 7 aa, Artificial Sequence
  Description of Artificial Sequence Synthetic peptide
KASSLES

SEQ ID NO: 6 - protein, 8 aa, Artificial Sequence
  Description of Artificial Sequence Synthetic peptide
KQYADYWT

SEQ ID NO: 7 - protein, 122 aa, Artificial Sequence
  Description of Artificial Sequence Synthetic polypeptide
EVQLVESGGGLVQPGGSLRLSCAASGFTFSSHDMHWVRQATGKGLEWVSGIGTAGDTYYPDSVKGRFTISRENAKNSLYLQMNSLRAGDTAVYYCARDRYSPTGHYYGMDVWGQGTTVTVSS

SEQ ID NO: 8 - protein, 106 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCKQYADYWTFGQGTKVEIK

SEQ ID NO: 9 - protein, 452 aa, Artificial Sequence
  Description of Artificial Sequence Synthetic polypeptide
EVQLVESGGGLVQPGGSLRLSCAASGFTFSSHDMHWVRQATGKGLEWVSGIGTAGDTYYPDSVKGRFTISRENAKNSLYLQMNSLRAGDTAVYYCARDRYSPTGHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 212 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCKQYADYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 11 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN